(19) United States
(12) Patent Application Publication
(10) Pub. No.: US 2022/0409711 A1
(43) Pub. Date: Dec. 29, 2022

(54) HLA RESTRICTED HORMAD1 T CELL RECEPTORS AND USES THEREOF

(71) Applicant: BOARD OF REGENTS, THE UNIVERSITY OF TEXAS SYSTEM, Austin, TX (US)

(72) Inventors: Cassian YEE, Houston, TX (US); Ke PAN, Houston, TX (US)

(73) Assignee: BOARD OF REGENTS, THE UNIVERSITY OF TEXAS SYSTEM, Austin, TX (US)

(21) Appl. No.: 17/774,298

(22) PCT Filed: Nov. 5, 2020
(86) PCT No.: PCT/US2020/059178

§ 371 Date: May 4, 2022

Related U.S. Application Data
(60) Provisional application No. 62/930,892, filed on Nov. 5, 2019.

(51) Int. Cl. A61K 39/00; C07K 14/47; C12N 5/0783; C07K 14/725; C07K 16/28; A61P 35/00; A61K 45/06

(52) U.S. Cl. CPC A61K 39/001184; C07K 14/4748; C12N 5/0638; C07K 14/7051; C07K 16/2809; A61P 35/00; A61K 45/06; A61K 2039/5154; C12N 2502/1121; C07K 2317/622

(57) ABSTRACT

Provided are T cell receptors (TCR) and TCR variable regions that can selectively bind a Hormad1 peptide/MHC complex. The TCR may be utilized in various therapies, such as autologous Hormad1-TCR adoptive T cell therapy to treat a cancer, such as a solid tumor expressing Hormad1. Methods for expanding related populations of T cells are provided.

This application claims benefit of priority of U.S. Provisional Patent Application No. 62/930,892 filed Nov. 5, 2019, which is hereby incorporated by reference in its entirety.

### BACKGROUND OF THE INVENTION

### 1. Field of the Invention

The present invention relates generally to the field of immunology and medicine. More particularly, it concerns antigenic peptides and recombinant T cell receptors (TCRs). In some embodiments the TCRs may be used to treat a cancer.

### 2. Description of Related Art

Although T cell-based therapies have shown promise for treating a variety of cancers, relapse after administration of an immunotherapy or chemotherapeutic remains a significant clinical problem. While aggressive B-cell non-Hodgkin lymphomas (NHL) and chronic lymphocytic leukemias (CLL) are often responsive to combinations of chemotherapy and anti-CD20 monoclonal antibodies (Plosker and Figgitt, 2003), about a third of patients experience recurrent relapses and eventually die of their disease (Chao M P, 2013). Recent studies with chimeric antigen receptor (CAR)-modified T cell therapy targeting CD19 resulted in complete remission (CR) rates of between 60 and 90% of patients with refractory B-cell malignancies (Porter et al., 2011; Kochenderfer et al., 2015; Turtle et al., 2016a; Neelapu et al., 2017; Schuster et al., 2015; Turtle et al., 2016b; Locke et al., 2017). In addition, a subset of these patients experienced long-term remissions, supporting the idea that adoptive T cell therapy can be used as an effective treatment and may be curative in some patients. Nonetheless, more than half of patients treated relapsed after the CD19 CAR T cell therapy, largely due to loss of CD19 expression on the tumor (Sotillo et al., 2015; Topp et al., 2014; Neelapu et al., 2017). Clearly, there is a need for novel targets for adoptive T cell therapeutic approaches to further improve clinical outcomes.

### SUMMARY OF THE INVENTION

The present disclosure, in some aspects, overcomes limitations in the prior art by providing Hormad1 peptides (e.g., SEQ ID NO:5) that are recognized by HLA-A2, as well as T cell receptors (TCRs) that can bind the Hormad1 peptide/MHC I complex. The peptides and TCR may be used, e.g., in an adoptive T cell therapy or in a soluble T cell therapy to treat a cancer.

An aspect of the present disclosure relates to an isolated Hormad1 peptide of 35 amino acids in length or less comprising SEQ ID NO:5, an amino acid sequence with at least 85% sequence identity to SEQ ID NO:5, an amino acid sequence comprising at least 6 contiguous amino acids of SEQ ID NO:5, or comprising an amino acid sequence that has only one substitution mutation relative to SEQ ID NO:5.

In some embodiments, the peptide comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:5. In some embodiments, the peptide comprises an amino acid sequence comprising at least 5, 6, 7, 8, or 9 contiguous amino acids of SEQ ID NO:5.

The peptide may be less than 30 amino acids, more preferably less than 29 amino acids, more preferably less than 28 amino acids, more preferably less than 27 amino acids, more preferably less than 26 amino acids, more preferably less than 25 amino acids, more preferably less than 24 amino acids, more preferably less than 23 amino acids, more preferably less than 22 amino acids, more preferably less than 21 amino acids, more preferably less than 20 amino acids, less than 19 amino acids, less than 18 amino acids, less than 17 amino acids, less than 16 amino acids, less than 15 amino acids, less than 14 amino acids, less than 13 amino acids, less than 12 amino acids, less than 11 amino acids, or less than 10 amino acids in length. In some embodiments, the peptide consists of SEQ ID NO: 5. The peptide may be further defined as an immunogenic peptide and/or a peptide that is capable of inducing cytotoxic T lymphocytes (CTLs) and selectively binds to HLA-A2. The term immunogenic may refer to the production of an immune response, such as a protective immune response. In some embodiments, the peptide is modified. In some embodiments, the modification comprises conjugation to a molecule. The molecule may be an antibody, a lipid, an adjuvant, or a detection moiety (tag).

Another aspect of the present disclosure relates to a pharmaceutical composition comprising the isolated peptide as described herein or above (e.g., SEQ ID NO:5) and a pharmaceutical carrier. The pharmaceutical composition may be formulated for parenteral administration, intravenous injection, intramuscular injection, or subcutaneous injection. In some embodiments, the pharmaceutical composition comprises a liposome, lipid-containing nanoparticle, or a lipid-based carrier. In some embodiments, the pharmaceutical preparation is formulated for injection. In some embodiments, the pharmaceutical preparation is formulated for inhalation. The pharmaceutical preparation may comprises or consists of a nasal spray.

Yet another aspect of the present disclosure relates to an isolated nucleic acid encoding the Hormad1-derived peptide as described herein or above (e.g., SEQ ID NO:5).

Another aspect of the present disclosure relates to a vector comprising the nucleic acid described herein or above.

Also provided is an isolated host cell comprising nucleic acids, peptides, TCRs, and vectors of the disclosure.

A further aspect relates to a method of making a cell comprising transferring a nucleic acid or vector of the disclosure into the cell.

Yet another aspect of the present disclosure relates to a method of stimulating an immune response in a mammalian subject, comprising administering an effective amount of the peptide described herein or above (e.g., SEQ ID NO:5) to the subject. In some embodiments, the peptide induces, activates, or stimulates the proliferation of Hormad1-specific T cells in the subject. The subject may have a cancer such as, e.g., a breast cancer, a lung cancer, bone cancer, endometrial cancer, hematopoietic or lymphoid cancer, gastrointestinal cancer, ovarian cancer, skin cancer, neuroblastoma, testicular cancer, thymoma, bladder cancer, uterine carcinoma, melanoma, sarcoma, cervix cancer, or head and neck cancer. It is also contemplated that a cancer described herein, such as breast cancer, a lung cancer, bone cancer, endometrial cancer, hematopoietic or lymphoid cancer, gastrointestinal cancer, ovarian cancer, skin cancer, neuroblastoma, testicular cancer, thymoma, bladder cancer, uterine carcinoma, melanoma, sarcoma, cervix cancer, or head and neck cancer may be excluded from the methods of the disclosure. The cancer may comprise a cancer that is positive for expression of the peptide. In some embodiments, the subject has been determined to have cells that are positive for the expression or overexpression of the peptide. In some embodiments, the method further comprises administering autologous dendritic cells to the subject, wherein the peptide is bound to or presented by the autologous dendritic cells. In some embodiments, the peptide and artificial antigen presenting cells (aAPCs) are administered to the subject, wherein the peptide is bound to or presented by the aAPCs. In some embodiments, the peptide is operatively linked to the artificial antigen presenting cells (aAPCs). The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. In some embodiments, the subject is a human. In some embodiments, the method further comprises administering at least a second anti-cancer therapy. The second anti-cancer therapy may be selected from the group consisting of a chemotherapy, a radiotherapy, an immunotherapy, or a surgery.

Another aspect of the present disclosure relates to a method of activating or expanding Hormad1-specific T cells comprising: (a) obtaining a starting population of cells from a mammalian subject and preferably from a blood sample from the mammalian subject, wherein the starting population of cells comprises T cells; and (b) contacting the starting population of cells ex vivo with the Hormad1-derived peptide as described herein or above (e.g., SEQ ID NO:5), thereby activating, stimulating proliferation, and/or expanding Hormad1-specific T cells in the starting population. In some embodiments, contacting is further defined as co-culturing the starting population of T cells with antigen presenting cells (APCs), wherein the APCs can present the Hormad1-derived peptide on their surface. In some embodiments, the APCs are dendritic cells. In some embodiments, the dendritic cells are autologous dendritic cells obtained from the mammalian subject. In some embodiments, contacting is further defined as co-culturing the starting population of T cells with artificial antigen presenting cells (aAPCs). In some embodiments, the artificial antigen presenting cells (aAPCs) comprise or consist of poly (lactide-co-glycolide) (PLGA), K562 cells, paramagnetic beads coated with CD3 and CD28 agonist antibodies, beads or microparticles coupled with an HLA-dimer and anti-CD28, or nanosize-aAPCs (nano-aAPC) that are preferably less than 100 nm in diameter. In some embodiments, the T cells are CD8⁺ T cells or CD4⁺ T cells. In some embodiments, the T cells are cytotoxic T lymphocytes (CTLs). In some embodiments, the starting population of cells comprises or consists of peripheral blood mononuclear cells (PBMCs). In some embodiments, the method further comprises isolating or purifying the T cells from the peripheral blood mononuclear cells (PBMCs). In some embodiments, the mammalian subject is a human. The method may further comprise reinfusing or administering the activated or expanded Hormad1-specific T cells to the subject.

Yet another aspect of the present invention relates to a Hormad1-specific T cell activated or expanded according to the methods described herein or above.

Another aspect of the present invention relates to a pharmaceutical composition comprising the Hormad1-specific T cells activated or expanded according to the methods described herein or above.

Yet another aspect of the present disclosure relates to an engineered T cell receptor (TCR) having antigenic specificity for Hormad1 or SEQ ID NO: 5, wherein the TCR comprises the amino acid sequences of SEQ ID NO: 6, 7, 8, 9, 10, and/or 11. The engineered TCR may comprise a TCR α CDR3 comprising an amino acid sequence with at least 90% sequence identity to SEQ ID NO:8 and a TCR β CDR3 comprising an amino acid sequence with at least 90% sequence identity to SEQ ID NO:11. The engineered TCR may comprise a TCR α CDR3 comprising an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:8 and a TCR β CDR3 comprising an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:11. In some embodiments, the TCR comprises a TCR α CDR1 and/or CDR2 comprising an amino acid sequence with at least 90% sequence identity to SEQ ID NO:6 and/or 7, respectively and a TCR β CDR1 and/or CDR2 comprising an amino acid sequence with at least 90% sequence identity to SEQ ID NO:9 and/or 10, respectively. In some embodiments, the TCR comprises a TCR α CDR1 and/or CDR2 comprising an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:6 and/or 7, respectively and a TCR β CDR1 and/or CDR2 comprising an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:9 and/or 10, respectively. In some embodiments, the engineered TCR comprises: (i) an α chain variable region having the amino acid sequence of SEQ ID NO:13 or 2, or a sequence having at least 90% sequence identity to SEQ ID NO: 13 or 2; and/or (ii) a β chain variable region having the amino acid sequence of SEQ ID NO: 15 or 4, or a sequence having at least 90% sequence identity to SEQ ID NO: 15 or 4. The engineered TCR may bind SEQ ID NO:5 when bound to HLA-A2. The engineered TCR may bind a MHC/peptide complex of SEQ ID NO:5 bound to HLA-A2. In some embodiments, the TCR comprises an α chain variable region having at least 95% identity to the amino acid sequence of SEQ ID NO: 13 or 2, and/or a β chain variable region having at least 95% identity to the amino acid sequence of SEQ ID NOs: 15. In some embodiments, the TCR comprises an a chain variable region having at least 99% identity to the amino acid sequence of SEQ ID NO: 13 or 2, and/or a β chain variable region having at least 95% identity to the amino acid sequence of SEQ ID NO: 15. In some embodiments, the TCR comprises an α chain variable region having at least 95% identity to the amino acid sequence of SEQ ID NO: 13 or 2, and/or a β chain having at least 99% identity to the amino acid sequence of SEQ ID NO: 15 or 4. In some embodiments, the TCR comprises an a chain variable region of SEQ ID NO: 13 or 2, and a β chain of SEQ ID NO: 15 or 4. In some embodiments, the soluble TCR is further defined as a single-chain TCR (scTCR), wherein the α chain and the β chain are covalently attached via a flexible linker. In some embodiments, the TCR comprises or consists of a bispecific TCR. The bispecific TCR may comprise an scFv that targets or selectively binds CD3.

Another aspect of the present disclosure relates to a multivalent TCR complex comprising a plurality of TCRs as described herein or above. In some embodiments, the multivalent TCR comprises 2, 3, 4 or more TCRs associated with one another. In some embodiments, the multivalent TCR is present in a lipid bilayer, in a liposome, or attached to a nanoparticle. In some embodiments, the TCRs are associated with one another via a linker molecule or a non-naturally occurring disulfide bond.

Yet another aspect of the present invention relates to a nucleic acid comprising or consisting of a nucleotide sequence encoding the TCR described herein or above. In some embodiments, the nucleic acid comprises a cDNA encoding the TCR.

Another aspect of the present disclosure relates an expression vector comprising the nucleic acid described above. The vector may comprise both the TCR α and TCR β genes on the same nucleic acid. In some embodiments, the nucleotide sequence encoding the TCR is under the control of a promoter. In some embodiments, the expression vector is a viral vector (e.g., a retroviral vector or a lentiviral vector).

Another aspect of the present invention relates to a host cell engineered to express the TCR described herein or above, preferably wherein the host cell comprises an expression vector described herein or above. In some embodiments, the cell is a T cell, NK cell, invariant NK cell, NKT cell, mesenchymal stem cell (MSC), or induced pluripotent stem (iPS) cell. In some embodiments, the host cell is an immune cell. In some embodiments, the host cell is isolated from an umbilical cord. In some embodiments, the T cell is a CD8+ T cell, CD4+ T cell, or γδ T cell. In some embodiments, the T cell is a regulatory T cell (Treg). In some embodiments, the cell is autologous. In some embodiments, the cell is allogeneic.

Yet another aspect of the present disclosure relates to a method for engineering the host cell as described above comprising contacting the immune cell with the nucleic acid as described herein or above or the expression vector as described herein or above. In some embodiments, the immune cell is a T cell or a peripheral blood lymphocyte. In some embodiments, the contacting is further defined as transfecting or transducing. The transfecting may comprise electroporating RNA encoding the TCR as described herein or above into the immune cell. The method may further comprise generating viral supernatant from the expression vector described herein or above to transducing the immune cell. In some embodiments, the immune cell is a stimulated lymphocyte (e.g., a human lymphocyte). In some embodiments, the stimulating comprises contacting the immune cell with or incubating the immune cell in OKT3 and/or IL-2. In some embodiments, the method further comprises sorting the immune cells to isolate TCR engineered T cells. The method may further comprise performing T cell cloning by serial dilution. In some embodiments, the method further comprises expansion of the T cell clone by the rapid expansion protocol.

Another aspect of the present disclosure relates to a method of treating cancer in a mammalian subject comprising administering an effective amount of the TCR-engineered cells as described herein or above to a subject, wherein the cancer expresses Hormad1. In some embodiments, the TCR-engineered cell is a T cell or peripheral blood lymphocyte. In some embodiments, T cell is a CD8+ T cell, CD4+ T cell, or Treg. In some embodiments, the cancer is a breast cancer, a lung cancer, esophagus carcinoma (esophageal cancer), bone cancer, endometrial cancer, hematopoietic or lymphoid cancer, gastrointestinal cancer, ovarian cancer, skin cancer, neuroblastoma, testicular cancer, thymoma, bladder cancer, uterine carcinoma, melanoma, sarcoma, cervix cancer, head or neck cancer. In some embodiments, the cancer is a solid tumor. The subject may be a human. In some embodiments, the TCR engineered cells are autologous or allogeneic to the subject. The method may further comprise lymphodepletion of the subject prior to administration of the Hormad1-specific T cells. In some embodiments, the lymphodepletion comprises administration of cyclophosphamide and/or fludarabine. The method may further comprise administering a second anticancer therapy to the subject. In some embodiments, the second therapy is a chemotherapy, immunotherapy, surgery, radiotherapy, or biological therapy. In some embodiments, the TCR-engineered cells, and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion. In some embodiments, the subject is determined to have or diagnosed as having cancer cells that overexpress Hormad1.

In some aspects, methods are provided for the treatment of cancer (e.g., a breast cancer, a lung cancer, etc.) comprising immunizing a subject with a purified tumor antigen or an immunodominant tumor antigen-specific peptide such as a Hormad1 peptide (SEQ ID NO:5). In some embodiments, the peptide can be injected in a solution (e.g., a saline solution) as a vaccine or to cause an immune response against the peptide. For example, in order to enhance the solubility of peptide and/or increase the immune response in the subject, an adjuvant can be included in the formulation or solution (e.g., Massarelli et al. 2019). Peptide pulsed mature dendritic cells can be administered to the subject in some embodiments. Approaches that may be used to cause an immune response or anti-cancer response against the peptide in a subject include, e.g., Wen et al. (2019) and Massarelli et al. (2019). In some embodiments, the Hormad1 peptide (SEQ ID NO:5) is bound to or presented by autologous dendritic cells that can be reinfused to a subject or human patient.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1D: Expression of Hormad1 in normal and tumor tissues. (FIG. 1A) Expression of Hormad1 in normal tissues. (FIG. 1B) High Hormad1 expression in esophageal cancer, lung cancer, and head and neck cancer. (FIG. 1C) High Hormad1 expression in cervical cancer, bladder cancer, and acute myeloid cancer. (FIG. 1D) High Hormad1 expression in melanoma and gastric cancer.

FIG. 2: T cell receptor (TCR) repertoire analysis for the Hormad1-56 A12 CTL cell line. The TCR α chain and β chain were cloned out from Hormad1-56 A12 CTL using 5′-RACE PCR. Both the α chain and the β chain were sequenced, and the sequences were annotated using the IMGT/V-QUEST tool. The TCR usage and the CDR3 sequence of a chain and β chain are shown.

FIG. 3: Hormad1-56 antigen-specific T cell receptor engineered T cell (TCR-T) generation. The full length TCR α chain and β chains were inserted into the retroviral vector pMSGV3 and then the recombinant retroviral vector was used to infect peripheral blood mononuclear cells (PBMCs). The empty retroviral vector was used as a control. After infection, the CD8+/Tetramer+ population was observed with flow cytometry (FCM) detection. After tetramer-guided sorting and expansion, a high purity of TCR-T cells was generated.

FIGS. 4A-4F: Hormad1-56 TCR-T cell killing assay with different targets. (FIG. 4A) Peptide titration assay: T2 cells were pulsed with various concentrations of Hormad1-56 peptide as the target. The effector to target (E:T) ratio was 20:1. (FIG. 4B-F) Tumor target killing assay: (FIG. 4B) Tumor cell line H1395 (HLA-A2+, Hormad1+) and H522 (HLA-A2+, Hormad1-), (FIG. 4C) Tumor cell line H1299 (HLA-A2-, Hormad1+) and H1299-A2 (HLA-A2 forced expressing, Hormad1+), (FIG. 4D) Tumor cell line H1355 (HLA-A2+, Hormad1+) and H1755 (HLA-A2+, Hormad1-), (FIG. 4E) K562-A2 cell line with forced expression of eGFP control gene or Hormad1 gene, or (FIG. 4F) H522 tumor cell line with forced expression of eGFP control gene or Hormad1 gene, was co-cultured with Hormad1-56 TCR-T cells. For the tumor target killing assay, the effector to target (E:T) ratio was from 40:1 to 1.25:1. The lysis ability of Hormad1-56 TCR-T to different targets was detected with Cr51 release assay (CRA).

FIG. 5: Functional detection of Hormad1-56 TCR-T cells with intracellular cytokine staining (ICS) assay. The Hormad1-56 TCR-T cells were co-cultured with H522, H1395, H1755, H1355, DFC1032, HSAEC2-KT, H1299, H1299-A2, H522-eGFP, H522-Hormad1, K562-A2-eGFP, K562-A2-Hormad1 with E:T=10:1 ratio. After overnight co-culturing, the TCR pathway downstream activated markers CD137, CD69, IFN-γ and TNF-α were detected with an ICS assay. The level of CD137, CD69, IFN-γ and TNF-α of Hormad1-56 TCR-T cells were significantly enhanced when Hormad1-56 TCR-T cells were co-cultured with positive targets H1395, H1355, H1299-A2, H522-Hormad1, K562-A2-Hormad1 compared with negative control.

FIGS. 6A-6B: The full-length sequence of the Hormad1-TCR. (FIG. 6A) Hormad1 CTL A12 TCR (TRAV4*01 F, TRBV13*01 F) Alpha Chain whole sequence. (SEQ ID NO: 2) (FIG. 6B) Hormad1 CTL A12 TCR (TRAV4*01 F, TRBV13*01 F) Beta Chain whole sequence. (SEQ ID NO: 4) Blue: Signal peptide; Yellow: Viable region; Red: CDR1, CDR2, CDR3; Black: Constant region.

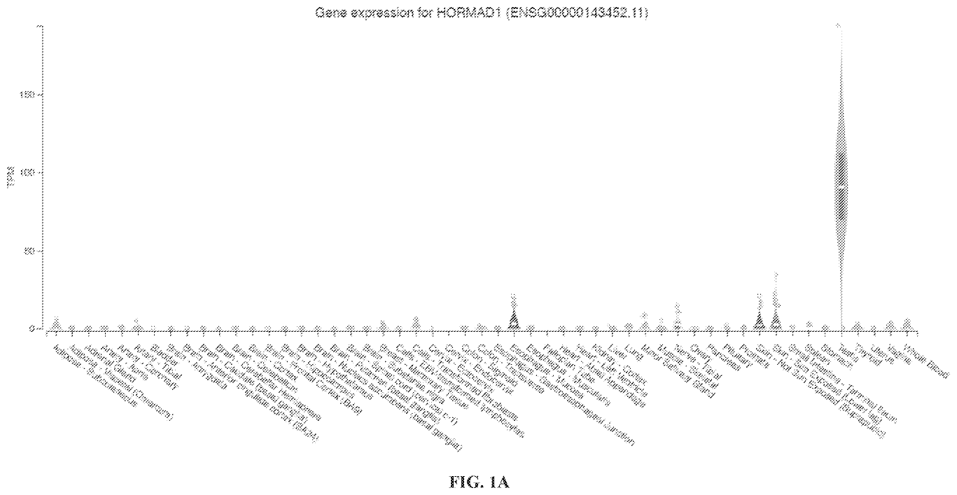

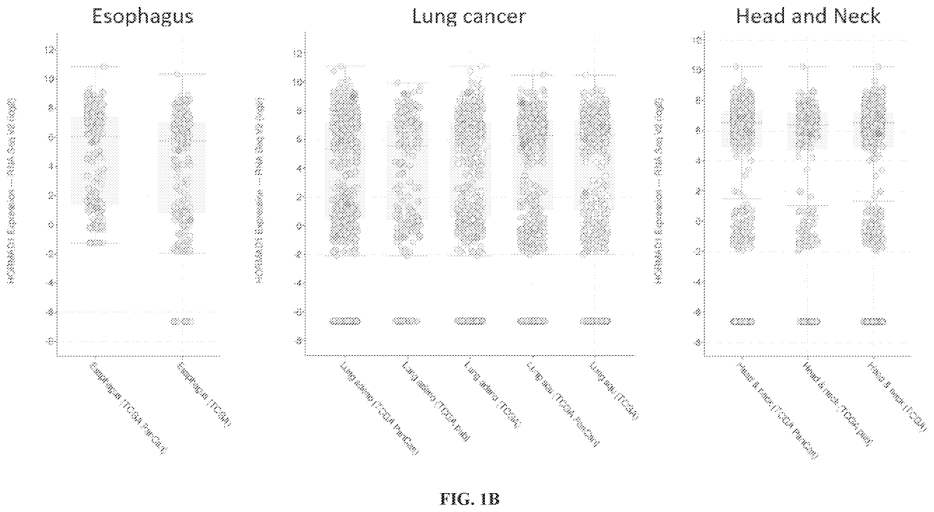

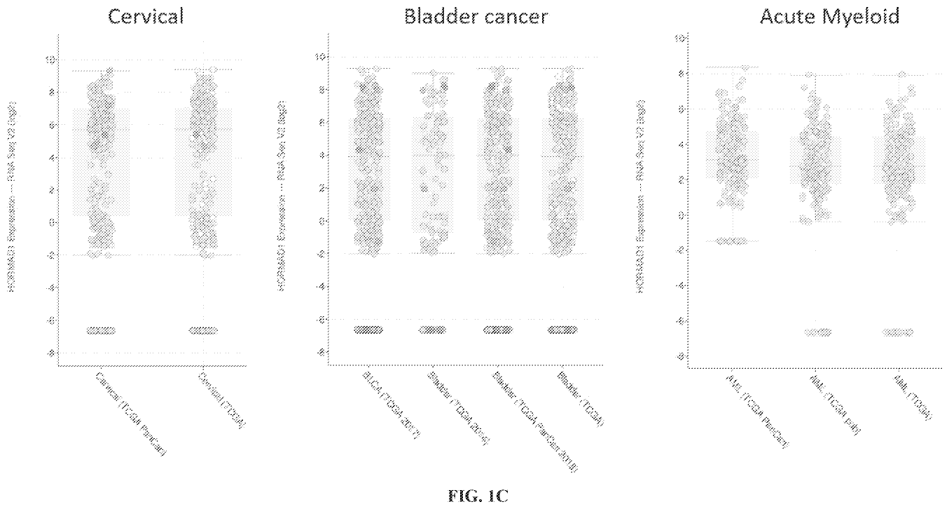

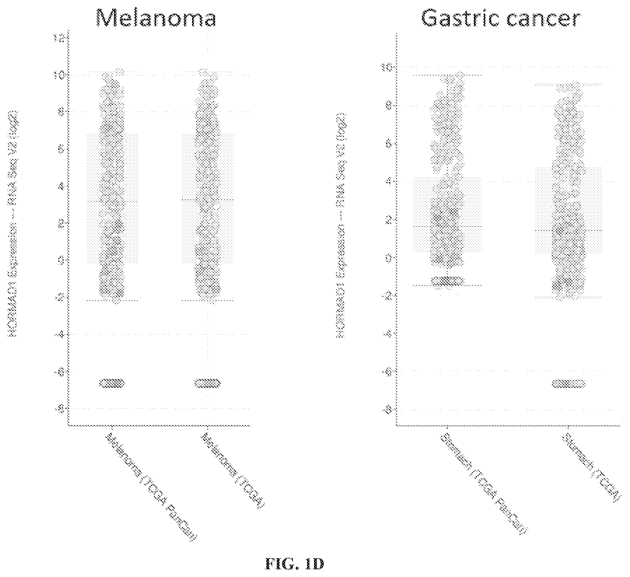

### DESCRIPTION OF ILLUSTRATIVE EMBODIMENTS

In some aspects, peptides derived from Hormad1 that are recognized by MHC I (HLA-A2) are provided and can be used in methods for the treatment of cancer. For example, the HLA-A2 restricted T cell epitope YLDDLCVKI (SEQ ID NO: 5) can be used to expand or activate antigen specific T cells in vitro. The expanded or activated antigen-specific T cells can be used in a cancer therapy, such as an adoptive cell transfer therapy. A variety of cancers that express Hormad1 may thus be treated in a mammalian subject (e.g., a human) such as, e.g., lung cancer, a cervical cancer, esophageal carcinoma, head and neck cancer, a leukemia, or solid tumors.

In additional aspects, cloned T cell receptor (TCR) sequences (e.g., SEQ ID NOs:1-4) that can bind the Hormad1-derived peptide/HLA-A2 complex are provided. A TCR of the present disclosure may be used to generate T cells that recognize the Hormad1-derived peptide/HLA-A2 complex. Such T cells include engineered T cells (TCR-T) that express the TCR. Those engineered T cells can be used to treat a cancer. Related soluble TCRs (sTCRs) and single chain TCRs (scTCRs) are also provided and can also be used to produce engineered T cells that can be utilized in an adoptive cell transfer therapy to treat a cancer.

The provided peptides and TCRs, or antigen binding domain or functional fragment of the TCR, can be included various additional constructs. For example, in some embodiments the antigen binding domain of the TCR can be included in a chimeric antigen receptor (CAR). The peptide (e.g., SEQ ID NO:5) can also be used to generate MHC-peptide multimers or tetramers (e.g., HLA-A2/peptide tetramers), and the peptide can be included in an immunogenic composition.

### I. ENGINEERED T CELL RECEPTORS

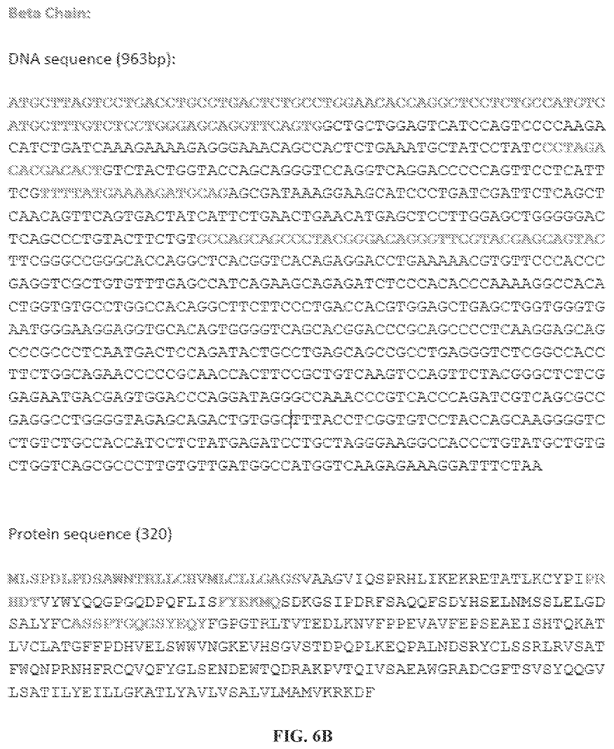

In various aspects, T cell receptors (TCRs) are provided that specifically bind a Hormad1-derived peptide (e.g., SEQ ID NO: 5)/MHC I (HLA-A2) complex. Thus, these TCRs can be used to target T cells to cancer cells that express Hormad1 protein. The antigen binding region of the TCR (such as CDR1, CDR2, and CDR3 as shown in FIGS. 6A-B) may be included in a soluble TCR (sTCR) or in a chimeric antigen receptor (CAR) as the extracellular domain comprising an antigen binding region. In some aspects, the TCR is an isolated or purified TCR. A polynucleotide encoding the TCR may be transfected into cells (e.g., autologous or allogeneic cells) that may be used in an adoptive cell transfer therapy, also referred to as an “adoptive cell therapy.”

In some embodiments, host cells such as, e.g., T cells (e.g., CD4⁺ T cells, CD8⁺ T cells, αβ T cells, γδ T cells, and Tregs), NK cells, invariant NK cells, NKT cells, mesenchymal stem cells (MSCs), or induced pluripotent stem (iPS) cells of the present disclosure can be genetically engineered to express receptors such as engineered TCRs and/or chimeric antigen receptors (CARs). For example, the autologous or allogeneic cells (e.g., isolated from an umbilical cord, or from a healthy donor) are modified to express a T cell receptor (TCR) having antigenic specificity for a short peptide derived from a cancer antigen (e.g., Hormad1 and SEQ ID NO:5), for example when presented in the context of a particular MHC allele (e.g., HLA-A2). In particular embodiments, the TCR has antigenic specificity for Hormad1-derived peptide (SEQ ID NO: 5)/HLA-A2 complex. In some embodiments, the engineered TCR comprises the CDR1, CDR2, and CDR3 regions of the TCRα and TCRβ chains, as shown in FIGS. 6A-B. In some embodiments, the engineered TCR has an α chain comprising an amino acid sequence having least 90, 91, 92, 93, 94, 95, 96, 97, 98, 99, or 100% sequence identity to SEQ ID NO:2 and/or a β chain comprising an amino acid sequence having at least 90, 91, 92, 93, 94, 95, 96, 97, 98, 99, or 100% sequence identity to SEQ ID NO:4. In some embodiments, the TCR has an a chain with at least 90, 91, 92, 93, 94, 95, 96, 97, 98, 99, or 100% sequence identity to SEQ ID NO: 1 and/or a β chain with at least 90, 91, 92, 93, 94, 95, 96, 97, 98, 99, or 100% sequence identity to SEQ ID NO: 3. Suitable methods of modifying the amino acid sequence (e.g., to introduce a substitution, deletion, or insertion mutation) are known in the art.

A. T Cell Receptors (TCRs)

In some aspects, recombinant T cell receptors (TCRs) are provided herein. A “T cell receptor” or “TCR” generally includes variable α and β chains (also known as TCRα and TCRP, respectively) or variable 7 and 6 chains (also known as TCRγ and TCRδ, respectively) and that are capable of specifically binding to an antigen peptide bound to an MHC receptor. In some embodiments, the TCR is in the αβ form, and referred to as a TCRαβ. In certain embodiments, the engineered TCR has an α chain variable region of SEQ ID NO: 2 and/or a β chain variable region of SEQ ID NO: 4. In some embodiments, the TCR α chain is encoded by a nucleic acid comprising or consisting of SEQ ID NO: 1, and the β chain is encoded by a nucleic acid comprising or consisting of SEQ ID NO: 3, respectively.

Embodiments of the disclosure relate to engineered T cell receptors. The term “engineered” refers to T cell receptors that have TCR variable regions grafted onto TCR constant regions to make a chimeric polypeptide that binds to peptides and antigens of the disclosure. In certain embodiments, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some embodiments, the TCR comprises non-TCR sequences. Accordingly, certain embodiments relate to TCRs with sequences that are not from a TCR gene. In some embodiments, the TCR is chimeric, in that it contains sequences normally found in a TCR gene, but contains sequences from at least two TCR genes that are not necessarily found together in nature.

The TCR provided below has been identified herein as selectively binding the Hormad1-derived peptide (e.g., SEQ ID NO: 5)/HLA-A2 complex:

α Chain DNA sequence (SEQ ID NO: 1)
ATGAGGCAAGTGGCGAGAGTGATCGTGTTCCTGAC
CCTGAGTACTTTGAGCCTTGCTAAGACCACCCAGC
CCATCTCCATGGACTCATATGAAGGACAAGAAGTG
AACATAACCTGTAGCCACAACAACATTGCTACAAA
TGATTATATCACGTGGTACCAACAGTTTCCCAGCC
AAGGACCACGATTTATTATTCAAGGATACAAGACA
AAAGTTACAAACGAAGTGGCCTCCCTGTTTATCCC
TGCCGACAGAAAGTCCAGCACTCTGAGCCTGCCCC
GGGTTTCCCTGAGCGACACTGCTGTGTACTACTGC
CTCGTGGGTGCGCGGGGAACTGCTCTGATCTTTGG
GAAGGGAACCACCTTATCAGTGAGTTCCAATATCC
AGAACCCTGACCCTGCCGTGTACCAGCTGAGAGAC
TCTAAATCCAGTGACAAGTCTGTCTGCCTATTCAC
CGATTTTGATTCTCAAACAAATGTGTCACAAAGTA
AGGATTCTGATGTGTATATCACAGACAAAACTGTG
CTAGACATGAGGTCTATGGACTTCAAGAGCAACAG
TGCTGTGGCCTGGAGCAACAAATCTGACTTTGCAT
GTGCAAACGCCTTCAACAACAGCATTATTCCAGAA
GACACCTTCTTCCCCAGCCCAGAAAGTTCCTGTGA
TGTCAAGCTGGTCGAGAAAAGCTTTGAAACAGATA
CGAACCTAAACTTTCAAAACCTGTCAGTGATTGGG
TTCCGAATCCTCCTCCTGAAAGTGGCCGGGTTTAA
TCTGCTCATGACGCTGCGGCTGTGGTCCAGCTAA
α chain protein sequence (SEQ ID NO: 2):
MRQVARVIVFLTLSTLSLAKTTQPISMDSYEGQEV
NITCSHNNIATNDYITWYQQFPSQGPRFIIQGYKT
KVTNEVASLFIPADRKSSTLSLPRVSLSDTAVYYC
LVGARGTALIFGKGTTLSVSSNIQNPDPAVYQLRD
SKSSDKSVCLFTDFDSQTNVSQSKDSDVYITDKTV
LDMRSMDFKSNSAVAWSNKSDFACANAFNNSIIPE
DTFFPSPESSCDVKLVEKSFETDTNLNFQNLSVIG
FRILLLKVAGFNLLMTLRLWSS
β chain DNA sequence (SEQ ID NO: 3):
ATGCTTAGTCCTGACCTGCCTGACTCTGCCTGGAA
CACCAGGCTCCTCTGCCATGTCATGCTTTGTCTCC
TGGGAGCAGGTTCAGTGGCTGCTGGAGTCATCCAG
TCCCCAAGACATCTGATCAAAGAAAAGAGGGAAAC
AGCCACTCTGAAATGCTATCCTATCCCTAGACACG
ACACTGTCTACTGGTACCAGCAGGGTCCAGGTCAG
GACCCCCAGTTCCTCATTTCGTTTTATGAAAAGAT
GCAGAGCGATAAAGGAAGCATCCCTGATCGATTCT
CAGCTCAACAGTTCAGTGACTATCATTCTGAACTG
AACATGAGCTCCTTGGAGCTGGGGGACTCAGCCCT
GTACTTCTGTGCCAGCAGCCCTACGGGACAGGGTT
CGTACGAGCAGTACTTCGGGCCGGGCACCAGGCTC
ACGGTCACAGAGGACCTGAAAAACGTGTTCCCACC
CGAGGTCGCTGTGTTTGAGCCATCAGAAGCAGAGA
TCTCCCACACCCAAAAGGCCACACTGGTGTGCCTG
GCCACAGGCTTCTTCCCTGACCACGTGGAGCTGAG
CTGGTGGGTGAATGGGAAGGAGGTGCACAGTGGGG
TCAGCACGGACCCGCAGCCCCTCAAGGAGCAGCCC
GCCCTCAATGACTCCAGATACTGCCTGAGCAGCCG
CCTGAGGGTCTCGGCCACCTTCTGGCAGAACCCCC
GCAACCACTTCCGCTGTCAAGTCCAGTTCTACGGG
CTCTCGGAGAATGACGAGTGGACCCAGGATAGGGC
CAAACCCGTCACCCAGATCGTCAGCGCCGAGGCCT
GGGGTAGAGCAGACTGTGGCTTTACCTCGGTGTCC
TACCAGCAAGGGGTCCTGTCTGCCACCATCCTCTA
TGAGATCCTGCTAGGGAAGGCCACCCTGTATGCTG
TGCTGGTCAGCGCCCTTGTGTTGATGGCCATGGTC
AAGAGAAAGGATTTCTAA
β Chain protein sequence (SEQ ID NO: 4):
MLSPDLPDSAWNTRLLCHVMLCLLGAGSVAAGVIQ
SPRHLIKEKRETATLKCYPIPRHDTVYWYQQGPGQ
DPQFLISFYEKMQSDKGSIPDRFSAQQFSDYHSEL
NMSSLELGDSALYFCASSPTGQGSYEQYFGPGTRL
TVTEDLKNVFPPEVAVFEPSEAEISHTQKATLVCL
ATGFFPDHVELSWWVNGKEVHSGVSTDPQPLKEQP
ALNDSRYCLSSRLRVSATFWQNPRNHFRCQVQFYG
LSENDEWTQDRAKPVTQIVSAEAWGRADCGFTSVS
YQQGVLSATILYEILLGKATLYAVLVSALVLMAMV
KRKDF
HLA-A2-restricted peptide derived from
Hormadl (SEQ ID NO: 5):
YLDDLCVKI
α chain CDR1 peptide (SEQ ID NO: 6):
NIATNDY
α chain CDR2 peptide (SEQ ID NO: 7):
GYKTK
α chain CDR3 peptide (SEQ ID NO: 8):
LVGARGTALIF
β chain CDR1 peptide (SEQ ID NO: 9):
PRHDT
β chain CDR2 peptide (SEQ ID NO: 10):
FYEKMQ
β chain CDR3 peptide (SEQ ID NO: 11):
ASSPTGQGSYEQY
α chain variable region DNA sequence
(SEQ ID NO: 12):
CTTGCTAAGACCACCCAGCCCATCTCCATGGACTC
ATATGAAGGACAAGAAGTGAACATAACCTGTAGCC
ACAACAACATTGCTACAAATGATTATATCACGTGG
TACCAACAGTTTCCCAGCCAAGGACCACGATTTAT
TATTCAAGGATACAAGACAAAAGTTACAAACGAAG
TGGCCTCCCTGTTTATCCCTGCCGACAGAAAGTCC
AGCACTCTGAGCCTGCCCCGGGTTTCCCTGAGCGA
CACTGCTGTGTACTACTGCCTCGTGGGTGCGCGGG
GAACTGCTCTGATCTTTGGGAAGGGAACCACCTTA
TCAGTGAGTTCCAAT
α chain variable region protein
sequence (SEQ ID NO: 13):
LAKTTQPISMDSYEGQEVNITCSHNNIATNDYITW
YQQFPSQGPRFIIQGYKTKVTNEVASLFIPADRKS
STLSLPRVSLSDTAVYYCLVGARGTALIFGKGTTL
SVSSN
β chain variable region DNA
sequence (SEQ ID NO: 14):
GCTGCTGGAGTCATCCAGTCCCCAAGACATCTGAT
CAAAGAAAAGAGGGAAACAGCCACTCTGAAATGCT
ATCCTATCCCTAGACACGACACTGTCTACTGGTAC
CAGCAGGGTCCAGGTCAGGACCCCCAGTTCCTCAT
TTCGTTTTATGAAAAGATGCAGAGCGATAAAGGAA
GCATCCCTGATCGATTCTCAGCTCAACAGTTCAGT
GACTATCATTCTGAACTGAACATGAGCTCCTTGGA
GCTGGGGGACTCAGCCCTGTACTTCTGTGCCAGCA
GCCCTACGGGACAGGGTTCGTACGAGCAGTACTTC
GGGCCGGGCACCAGGCTCACGGTCACA
β chain variable region protein
sequence (SEQ ID NO: 15):
AAGVIQSPRHLIKEKRETATLKCYPIPRHDTVYWY
QQGPGQDPQFLISFYEKMQSDKGSIPDRFSAQQFS
DYHSELNMSSLELGDSALYFCASSPTGQGSYEQYF
GPGTRLTVT

Unless otherwise stated, the term “TCR” should be understood to encompass both full-length native TCR polypeptides, as well as functional fragments thereof in various combinations, including the αβ form or γδ form. As used herein, a “functional” TCR or fragment thereof is capable of binding its cognate subunit (e.g., α binding β, or γ binding δ) to form a full-length or truncated TCR that remains capable of binding its cognate peptide presented in the context of an appropriate MHC allele (e.g., HLA-A2).

Thus, for purposes herein, reference to a TCR includes any TCR or a TCR fragment that can bind an antigenic peptide, such as an antigen-binding portion of a TCR that binds to a specific antigenic peptide bound in an MHC molecule (i.e. a MHC-peptide complex). The terms “antigen-binding portion” or “antigen-binding fragment” of a TCR are used interchangeably herein to refer to a molecule that contains a portion of a TCR that binds the antigen (e.g., a MHC-peptide complex) to which the full TCR binds.

The variable domains of TCR chains are generally understood to form loops, or complementarity determining regions (CDRs), analogous to those present in immunoglobulins which confer antigen recognition; in TCRs, the CDRs determine peptide specificity by forming the binding site of the TCR molecule. Typically, like immunoglobulins, the CDRs are separated by framework regions (FRs) (see, e.g., Jores et al., 1990; Chothia et al., 1988; see also Lefranc et al., 2003). CDR3 regions on the α and β chains of a TCR are generally understood to participate in binding a processed antigen peptide. In some embodiments, the variable region of the β-chain can contain a further hypervariability (HV4) region.

α/β and γ/δ TCRs are structurally similar, but T cells expressing them may have distinct anatomical locations or functions. As would be appreciated by one of skill in the applicable art, TCRs are found on the surface of T cells (or T lymphocytes) where it may recognize an antigen-derived peptide bound to major histocompatibility complex (MHC) molecules. TCRs contain different regions, including: a constant domain, a transmembrane domain and/or a short cytoplasmic tail (see, e.g., Janeway et al, Immunobiology: The Immune System in Health and Disease, 3^{rd} Ed., Current Biology Publications, p. 433, 1997). The TCR α and β chains can associate with invariant proteins of the CD3 complex involved in mediating signal transduction.

In some embodiments, the TCR comprises a functional fragment of a Hormad1-TCR. In some embodiments, the functional fragment comprises a constant domain and a variable domain of a Hormad1-TCR. Similar to immunoglobulins, the extracellular portion of TCR chains (e.g., α-chain, β-chain) can contain two immunoglobulin domains, a variable domain (e.g., Vₐ; typically amino acids 1 to 116 based on Kabat numbering Kabat et al., “Sequences of Proteins of Immunological Interest,” US Dept. Health and Human Services, Public Health Service National Institutes of Health, 1991, 5^{th} ed.) at the N-terminus, and one constant domain (e.g., α-chain constant domain or Ca, typically amino acids 117 to 259 based on Kabat, β-chain constant domain, typically amino acids 117 to 295 based on Kabat) adjacent to the cell membrane. For example, in some cases, the extracellular portion of the TCR formed by the two chains (e.g., either αβ form or γδ form) contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, it may be possible to improve TCR gene transfer by adding a single cysteine on each receptor chain to promote the formation of an additional interchain disulfide bond, e.g., as described in Cohen et al. (2007).

A CDR may also comprise 1, 2, 3, 4, 5, 6, 7, 8, 9, 10, 11, 12, 13, 14, 15, 16, 16, 18, 19, 20, 21, 22, 23, or more contiguous amino acid residues (or any range derivable therein) flanking one or both sides of a particular CDR sequence in the context of the variable region of the TCR-a or TCR-b polypeptide; therefore, there may be one or more additional amino acids at the N-terminal or C-terminal end of a particular CDR sequence, such as those shown in the variable regions of SEQ ID NOS:13 and 15. Alternatively, or in combination, a CDR may also be a fragment of a CDR described herein and may lack at least 1, 2, 3, 4, or 5 amino acids from the C-terminal or N-terminal end of a particular CDR sequence.

In some embodiments, the TCR chains each comprise a transmembrane domain. In some embodiments, the transmembrane domain is positively charged. In some cases, the TCR chains comprise a cytoplasmic tail. In some cases, the TCR can associate with other molecules like CD3. For example, a TCR containing constant domains and a transmembrane domain can anchor the protein in the cell membrane and enable it to associate with invariant subunits of the CD3 signaling apparatus or complex.

CD3 is a multi-subunit complex comprising distinct chains: γ, δ, ε, and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily. The transmembrane domains of the CD3γ, CD3δ, and CD3ε chains are negatively charged, which is a characteristic that allows these chains to associate with the positively charged T cell receptor chains. The intracellular tails of the CD3γ, CD3δ, CD3ε, and CD3ζ chains each contain a conserved motif known as an immunoreceptor tyrosine-based activation motif (ITAM). ITAM are conserved amino acid sequences that may be repeated and are involved in the signaling capacity or signal transduction of the TCR complex. These accessory molecules have negatively charged transmembrane domains and play a role in propagating the signal from the TCR into the cell. The CD3- and ζ-chains, together with the TCR, form what is known as the T cell receptor complex (TCR complex).

In some embodiments, the TCR comprises a heterodimer comprising one TCR α polypeptide and one TCR β polypeptide. A TCR may comprise a heterodimer comprising one TCR γ polypeptide and one TCR δ polypeptide. In some embodiments, the TCR comprises a single chain TCR (scTCR). In some embodiments, the polypeptides of the TCR heterodimer are covalently linked. In some embodiments, the covalent linkage is by one or more disulfide bonds. In some embodiments, the one or more disulfide bonds comprises a naturally occurring disulfide bond as found in a native TCR. In some embodiments, the one or more disulfide bonds comprise a non-naturally occurring disulfide bond not found in a native TCR.

TCRs of the present disclosure can be expressed in a cell, such as a T cell, by transfecting the cells with a nucleic acid encoding the TCR using a variety of methods, as would be appreciated by one of skill in the art. For example, viral vectors can be used to transfect T cells (e.g., Levine et al., 2017). In some embodiments, non-viral methods are used to transfect T cells (e.g., as described in Riet et al., 2013), including electro-transfection methods (e.g., Zhang et al., 2018).

B. Soluble TCRs

In some embodiments, the present disclosure provides soluble TCRs, which may include variable regions of a TCR specific for a Hormad1-derived peptide provided herein (e.g., SEQ ID NOs:13 and 15). Soluble TCRs are useful, not only for the purpose of investigating specific TCR-MHC interactions, but also potentially as a diagnostic tool to detect infection, or to detect autoimmune disease biomarkers. Soluble TCRs also have applications in staining, for example to stain cells for the presence of a particular peptide antigen presented in the context of the MHC. Similarly, soluble TCRs can be used to deliver a therapeutic agent, for example a cytotoxic compound or an immunostimulating compound, to cells presenting a particular antigen. Soluble TCRs may also be used to inhibit T cells, for example, those reacting to an auto-immune peptide antigen.

In the context of this application, “solubility” is defined as the ability of the TCR to be purified as a monodisperse heterodimer in phosphate buffered saline (PBS) (KCL 2.7 mM, KH₂PO₄ 1.5 mM, NaCl 137 mM and Na₂PO4 8 mM, pH 7.1-7.5. Life Technologies, Gibco BRL) at a concentration of 1 mg/ml and for more than 90% of said TCR to remain as a monodisperse heterodimer after incubation at 25° C. for 1 hour.

In some aspects, the present disclosure provides a soluble T cell receptor (sTCR) comprising (i) all or part of a TCR α chain (e.g., SEQ ID NO: 1 or 2), except the transmembrane domain thereof, and (ii) all or part of a TCR β chain (e.g., SEQ ID NO: 3 or 4), except the transmembrane domain thereof, wherein (i) and (ii) each comprise a functional variable domain and at least a part of the constant domain of the TCR chain, and are linked by a disulfide bond between constant domain residues which is not present in the native TCR. In some aspects, the soluble TCR comprises a TCR α or γ chain extracellular domain dimerized to a TCR β or δ chain extracellular domain respectively, by means of a pair of C-terminal dimerization peptides, such as leucine zippers (International Patent Publication No. WO 99/60120; U.S. Pat. No. 7,666,604).

In some embodiments, the entire antigen binding region including the variable regions of the TCR (e.g., see FIGS. 6A-B) can be included in the sTCR. The sTCR may be a single-chain T cell receptor (scTCR), wherein the variable regions from the α and β chains (Vₐ and VO) are covalently attached via a flexible linker, and the end of the variable region (typically the end of Vβ that is not attached to the linker) is covalently attached to a therapeutic compound (e.g., a toxin, a chemotherapeutic, etc.) or an imaging agent. sTCRs can recognize intracellular or extracellular epitopes when presented by a MHC, and sTCRs can be used for identification of natural peptide ligands in disease (e.g., Walseng et al., 2015; Boulter et al., 2005). Thus, the sTCRs may be administered to a subject, such as a human patient, to visualize tumor cells or to deliver a therapeutic compound to cancerous cells to treat the cancer. A variety of therapeutic molecules or toxins may be delivered by sTCRs to cells such as cancer cells that express the Hormad1-derived peptide/HLA-A2 complex, such as ¹³¹I, Auristatins, maytansines, calicheamicin, STING agonists, cytokines, chemokines, costimulatory agonists (e.g., OX40), or other chemotherapeutics. In this way, the sTCRs can be used for targeted delivery of therapeutic molecules to a tumor site. In some embodiments, the sTCRs comprise or are covalently attached to a fluorescent or radioactive probe.

A soluble TCR, which may be human or produced in human cells, of the present disclosure may be provided in substantially pure form, or as a purified or isolated preparation. For example, it may be provided in a form which is substantially free of other proteins.

A plurality of soluble TCRs of the present disclosure may be provided in a multivalent complex. Thus, the present disclosure provides, in one aspect, a multivalent T cell receptor (TCR) complex, which comprises a plurality of soluble T cell receptors as described herein. Each of the plurality of soluble TCRs is preferably identical. The multivalent TCRs may contain two or more ligand-binding TCRα/β subunits (e.g., see Schamel et al., 2005).

A multivalent TCR complex generally comprises a multimer of two or three or four or more T cell receptor molecules associated (e.g., covalently or otherwise linked) with one another, preferably via a linker molecule. Suitable linker molecules include, but are not limited to, multivalent attachment molecules such as avidin, streptavidin, neutravidin and extravidin, each of which has four binding sites for biotin. Thus, biotinylated TCR molecules can be formed into multimers of T cell receptors having a plurality of TCR binding sites. The number of TCR molecules in the multimer will depend upon the quantity of TCR in relation to the quantity of linker molecule used to make the multimers, and also on the presence or absence of any other biotinylated molecules. Preferred multimers are dimeric, trimeric or tetrameric TCR complexes.

TCR or multivalent TCR complexes may be attached to a membrane structure (e.g., liposomes) or solid structures that are preferably particles such as beads (e.g., latex beads). In some embodiments, the structures are coated with T cell receptor multimers rather than with individual T cell receptor molecules. In the case of liposomes, the T cell receptor molecules or multimers thereof may be attached to or otherwise associated with the membrane. Techniques for this are well known to those skilled in the art.

A label or another moiety, such as a toxic or therapeutic moiety, may be included in the multivalent TCR complex. For example, the label or other moiety may be included in a mixed molecule multimer. An example of such a multimeric molecule is a tetramer containing three TCR molecules and one peroxidase molecule. This may be achieved by mixing the TCR and the enzyme at a molar ratio of about 3:1 to generate tetrameric complexes and isolating the desired complex from any complexes not containing the correct ratio of molecules. These mixed molecules may contain any combination of molecules, provided that steric hindrance does not compromise or does not significantly compromise the desired function of the molecules. The positioning of the binding sites on the streptavidin molecule can be suitable for mixed tetramers since steric hindrance is not likely to occur.

In some embodiments, peptides provided herein (e.g., SEQ ID NO:5) can be used to generate MHC-peptide tetramers (e.g., HLA-A2/peptide tetramers). These tetramers can be used to isolate epitope-specific T cells (e.g., tumor infiltrating lymphocytes, or TILs) from patient samples, or in vitro after pulsing professional APCs with specific Hormad1 peptides, Hormad1 protein, or nucleotide sequences encoding specific Hormad1 peptides or Hormad1 protein. In some instances, the MHC-peptide tetramer can be used to visualize T cells in tissues (e.g., Dileepan et al., 2015). MHC multimer-guided methods can also be used to facilitate isolation of functional T cell receptors from single cells that may be used in an immunotherapy. For example, direct isolation of paired full-length TCR sequences from non-expanded antigen-specific T cells can be achieved using PCR-based T cell receptor single cell analysis methods (TCR-SCAN) (e.g., Dossinger et al., 2013). Thus, using a multimer guided sorting strategy, T cells that selectively identify a Hormad1 peptide (e.g., SEQ ID NO:5) can be isolated from HLA-A2 positive patients' PBMCs or from T cells that have been stimulated (e.g., using the peptide, or aAPCs). After infusion, the antigen:specific T cells can be tracked with the tetramer or multimer for the evaluation of long-term persistence in vivo.

The TCRs (or multivalent complexes thereof) of the present disclosure may alternatively or additionally be associated with (e.g. covalently or otherwise linked to) a therapeutic agent which may be, for example, a toxic moiety for use in cell killing, or an immunostimulating agent such as an interleukin or a cytokine. A multivalent TCR complex of the present disclosure may have enhanced binding capability for a TCR ligand compared to a non-multimeric T cell receptor heterodimer. Thus, the multivalent TCR complexes may be used in some embodiments for tracking or targeting cells presenting particular antigens in vitro or in vivo. The TCRs or multivalent TCR complexes may therefore be provided in a pharmaceutically acceptable formulation for use in vivo.

The present disclosure also provides a method for delivering a therapeutic agent to a target cell, which method comprises contacting potential target cells with a TCR or multivalent TCR complex under conditions to allow attachment of the TCR or multivalent TCR complex to the target cell, said TCR or multivalent TCR complex being specific for the TCR ligand and having the therapeutic agent associated therewith.

In some embodiments, the soluble TCR or multivalent TCR complex can be used to deliver therapeutic agents to the location of cells presenting a particular antigen. This can be useful, e.g., for the treatment of tumors. A therapeutic agent could be delivered such that it would exercise its effect locally and not only on the cell it binds (e.g., a chemotherapeutic, radioactive, or enzymatic agent may result in a local effect near or on a tumor). Thus, one particular strategy envisages anti-tumor molecules linked to T cell receptors or multivalent TCR complexes specific for tumor antigens.

Many therapeutic agents can be employed for this use, for instance radioactive compounds, enzymes (e.g., perforin) or chemotherapeutic agents (e.g., cisplatin). To reduce or limit toxic effects in the desired location the toxin may be provided inside a liposome linked to streptavidin so that the compound is released slowly. This may reduce damaging effects during transport in the body and may help to limit toxic effects until after binding of the TCR to the relevant antigen presenting cells or cells (e.g., cancerous cells) that express the Hormad1 antigen.

Other suitable therapeutic agents include: (1) small molecule cytotoxic agents, i.e. compounds with the ability to kill mammalian cells having a molecular weight of less than 700 daltons. Such compounds could also contain toxic metals capable of having a cytotoxic effect. Furthermore, it is to be understood that these small molecule cytotoxic agents also include pro-drugs, i.e., compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetrexate glucuronate, auristatin E vincristine and doxorubicin; (2) peptide cytotoxins, i.e. proteins or fragments thereof with the ability to kill mammalian cells. Examples include ricin, diphtheria toxin, Pseudomonas bacterial exotoxin A, DNAase and RNAase; (3) radio-nuclides, i.e. unstable isotopes of elements which decay with the concurrent emission of one or more of a or β particles, or 7 rays. Examples include iodine 131 (¹³¹I) rhenium 186 (¹⁸⁶Re), indium 111 (¹¹¹In), yttrium 90 (⁹⁰Yt), bismuth 210 and 213 (²¹⁰Bi and ²¹³Bi), actinium 225 (²²⁵Ac), and astatine 213 (²¹³At); (4) prodrugs, such as antibody directed enzyme pro-drugs; and (5) immuno-stimulants, i.e. moieties which stimulate immune response. Examples include cytokines such as IL-2, chemokines such as IL-8, platelet factor 4, melanoma growth stimulatory protein, etc., antibodies or fragments thereof such as anti-CD3 antibodies or fragments thereof, complement activators, xenogeneic protein domains, allogeneic protein domains, viral/bacterial protein domains and viral/bacterial peptides.

The soluble TCRs of the present disclosure may be used to modulate T cell activation by binding to a specific TCR ligand and thereby inhibiting T cell activation. Autoimmune diseases involving T cell-mediated inflammation and/or tissue damage (e.g., type I diabetes) may be treated using this approach. Knowledge of the specific peptide epitope presented by the relevant pMHC is required for this use.

The soluble TCRs and/or multivalent TCR complexes of the present disclosure may be used in the preparation of a composition for the treatment of cancer or autoimmune disease.

Also provided are methods of treating cancer (e.g., a leukemia, lung cancer, esophagus carcinoma, head and neck cancer, or cervical cancer, etc.) or other cancer that expresses Hormad1 as described herein) or autoimmune disease comprising administration to a patient in need thereof of an effective amount of the soluble TCRs and/or multivalent TCR complexes of the present invention.

As is common in anti-cancer and autoimmune therapies, the TCRs of the present disclosure may be used in combination with other agents for the treatment of cancer or an autoimmune disease, and one or more additional therapeutic or therapy may be administered to treat other related condition(s) found in the patient groups.

C. Bispecific TCR

In some embodiments, TCRs of the present disclosure are included in a bispecific T cell receptor (TCR). Bispecific TCRs generally comprise a TCR that is fused to, ligated to, or covalently bonded to either an scFv or an antibody (e.g., McCromack et al., 2013). In some embodiments, the bispecific TCRs of the present disclosure comprise a Hormad1-directed TCR and a T cell recruiting antibody domain or scFv (e.g., a scFv directed against CD3 or other immuno-modulating T cell surface protein). Bispecific TCRs may allow T cells to become activated and attack the tumor, regardless of the T cells' intrinsic specificity. Bispecific platforms that can be used with the TCR of the present disclosure include TCER® molecules (Immatics, Houston, Tex.). Additional examples of bispecific TCR are ImmTACs (e.g., Oates et al., 2013).

D. Chimeric Antigen Receptors

Chimeric antigen receptors (CAR) are engineered receptors that can be expressed by T cells and can bind an antigen, such as an antigen on a cancer cell. CAR generally comprise different domains, including an antigen binding region domain, a transmembrane domain, and an endodomain. Upon antigen recognition, the endodomain transmits activation and costimulatory signals to the T cell. Chimeric antigen receptor molecules are non-naturally occurring and are distinguished by their ability to both bind antigen and transduce activation signals via immunoreceptor activation motifs (ITAM's) present in their cytoplasmic endodomains. CAR T cells are T cells that have been genetically modified to express the CAR.

A soluble TCR construct can be fused to a CAR-signaling tail (i.e., the transmembrane domain and endodomain) to direct T cells to recognize an antigen, e.g., as described in Walseng et al. (2017). Such CAR constructs have been referred to as “TCR-CARs”. A CAR may thus comprise a TCR binding region (e.g., as shown in FIGS. 6A-B) or a soluble TCR of the present disclosure that is covalently linked to, or expressed as a fusion protein with, a transmembrane domain and an endodomain. The endodomain may comprise, e.g., CD3ζ, a CD28 intracellular signaling domain, 4-1BB (CD137), (CD3ζ and CD28), CD27, OX-40 (CD134), DAP10, or 4-1BB.

### II. ADOPTIVE CELL TRANSFER THERAPIES

Provided herein are methods for treating or delaying progression of cancer in an individual comprising administering to the individual an effective amount an antigen-specific immune or stem cell (e.g., autologous or allogeneic T cells (e.g., regulatory T cells, CD4+ T cells, CD8+ T cells, α-β T cells, or γ-δ T cells), NK cells, invariant NK cells, NKT cells, mesenchymal stem cells (MSCs), or induced pluripotent stem (iPS) cells) therapy, such as a Hormad1-specific cell therapy. Adoptive T cell therapies with genetically engineered TCR-transduced T cells (e.g., expressing a TCR comprising one or more of SEQ ID NOs:1-4, such as SEQ ID NOs: 2 and SEQ ID NOs: 4) are also provided herein. In some embodiments, the adoptive cell transfer therapy is provided to a subject (e.g., a human patient) in combination with a second therapy, such as a chemotherapy, a radiotherapy, a surgery, or a second immunotherapy.

Peptides provided herein (e.g., SEQ ID NO:5) can also be used to generate antigen specific cytotoxic T cell (CTL) cell lines or clones that can be used in an adoptive immunotherapy. The peptide, or a corresponding polynucleotide that encodes the peptide, can be loaded onto dendritic cells, lymphoblastoid cell lines (LCL), PBMC or artificial antigen presenting cells (aAPCs), and then co-cultured with T cells for several rounds of stimulation to generate antigen-specific CTL cell lines or clones (e.g., Neal et al., 2017). A variety of antigen presenting cells (APCs) may be used to expand T cells ex vivo, and various strategies for antigen loading of dendritic cells to enhance the antitumor response can be used (e.g., see Strome et al., 2002). The resulting autologous CTL cell lines or clones can be used in an adoptive cell transfer immunotherapy for the treatment of cancer patients.

Embodiments of the present disclosure comprise methods of obtaining autologous T cells from a subject, methods of making TCR-engineered immune or stem cells, and methods of administering TCR-engineered cells to a subject as an immunotherapy to target cancer cells. In particular, the TCR-engineered immune or stem cells (e.g., autologous or allogeneic T cells (e.g., regulatory T cells, CD4+ T cells, CD8+ T cells, α-β T cells, or γ-δ T cells), NK cells, invariant NK cells, NKT cells, mesenchymal stem cells (MSCs), or induced pluripotent stem (iPS) cells) cells are antigen-specific cells (e.g., Hormad1-specific cells). Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies” (e.g., Eshhar et al., 1995). These approaches have given rise to numerous protocols for T cell preparation and immunization which can be used in the methods described herein.

A. T Cell Preparation and Administration

In some embodiments, the engineered T cells are autologous (i.e., isolated from the patient to be treated). In some embodiments, the engineered T cells are allogeneic. In some embodiments, the allogeneic T cells comprise T cells pooled from multiple donors.

In some embodiments, the T cells are derived from blood, bone marrow, lymph, umbilical cord, or lymphoid organs. The T cells are most preferably human cells. In some embodiments, T cells obtained from cord blood can have improved antitumor properties as compared to T cells obtained from an adult donor (e.g., Hiwarkar et al., 2015). The cells typically are primary cells, such as those isolated directly from a subject and/or isolated from a subject and frozen. In some embodiments, the cells include one or more subsets of T cells or other cell types, such as T cells from whole-blood, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem (iPS) cells; for example, the stem cells or iPS cells may be differentiated into various T cell populations. In some embodiments, the methods include isolating cells from the subject, preparing, processing, culturing, and/or engineering them as described herein, and re-introducing them into the same patient (if they are autologous) or into a different patient (if they are allogeneic), before or after cryopreservation.

Among the sub-types and subpopulations of T cells (e.g., CD4+ and/or CD8+ T cells) are naive T (T_{N}) cells, effector T cells (T_{EFF}), memory T cells (T_{MEM}) and sub-types thereof, such as stem cell memory T (TSC_{M}), central memory T (TC_{M}), effector memory T (T_{EM}), or terminally differentiated effector memory T cells (T_{EM}RA), T cells from tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, α/β T cells, and δ/γ T cells.

In some embodiments, sub-populations of T cells can be generated by separating, enriching, or depleting cells that are positive or negative for a specific marker, such as a cell surface marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. A variety of methods may be used for separation of cells based on expression of markers, including magnetic activated cell sorting (MACS) and fluorescence activated cell sorting (FACS).

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (TC_{M}) cells is carried out to increase efficacy, such as to improve long-term survival, expansion, and/or engraftment following administration (e.g., see Terakura et al., 2012; Wang et al., 2012).

In some embodiments, the T cells are autologous T cells. In this method, a biological sample (e.g., a blood sample, or a bone marrow sample) is obtained from a patient. In some embodiments, a cell suspension or culture is prepared from a biological sample obtained from a patient (e.g., from a tumor). The single cell suspension can be obtained in any suitable manner, e.g., mechanically (e.g., disaggregating the tumor using, e.g., a gentleMACS™ Dissociator, Miltenyi Biotec, Auburn, Calif.) or enzymatically (e.g., using collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5-6 days, or 5-21 days, or 10-14 days.

In some embodiments, naked DNA or a suitable vector encoding a TCR or a CAR of the present disclosure can be introduced into a subject's T cells (e.g., T cells obtained from a human patient with cancer or other disease). Methods of stably transfecting T cells by electroporation using naked DNA are known in the art. See, e.g., U.S. Pat. No. 6,410,319. Naked DNA generally refers to the DNA encoding a chimeric receptor of the present invention contained in a plasmid expression vector in proper orientation for expression (e.g., Zhang et al., 2018). In some embodiments, the use of naked DNA may reduce the time required to produce T cells expressing a TCR generated via methods of the present invention. Transduction techniques described in Heemskerk et al., 2008 and Johnson et al., 2009, can be used. Electroporation of RNA coding for the full length TCR α and β (or γ and δ) chains can be used as alternative to overcome long-term problems with autoreactivity caused by pairing of retrovirally transduced and endogenous TCR chains. In some embodiments, non-viral RNA transfection may be used to transiently modify T cells, e.g., as described in Riet et al. (Methods Mol Biol. 2013; 969:187-201).

Alternatively, a viral vector (e.g., a retroviral vector, adenoviral vector, adeno-associated viral vector, or lentiviral vector) can be used to introduce the TCR or chimeric construct into T cells. Generally, a vector encoding a TCR or CAR that is used for transfecting a T cell from a subject should generally be non-replicating in the subject's T cells. A large number of vectors are known that are based on viruses, where the copy number of the virus maintained in the cell is low enough to maintain viability of the cell. Illustrative vectors include the pFB-neo vectors (STRATAGENE®) as well as vectors based on HIV, SV40, EBV, HSV, or BPV.

In some embodiments, a TCR nucleotide sequence (e.g., a DNA or RNA sequence) encoding an a chain and a β chain of the disclosure (e.g., see FIGS. 6A-B; SEQ ID NOs 1-4) can be cloned into a retrovirus, lentivirus, or other expression vector, such as the MSCV (murine stem cell virus) or plasmid (e.g., adeno-associated virus-derived plasmid). T cells can be genetically altered to express the TCR. PBMCs are a source of both antigen-presenting cells and T cells. The TCR-expressing T cells can be used in an adoptive cell transfer therapy for cancer patients.

Once it is established that the transfected or transduced T cell is capable of expressing a TCR or CAR as a surface membrane protein and at a desired level, it can be determined whether the TCR or chimeric receptor is functional in the host cell to provide for the desired signal induction. Subsequently, the transduced T cells may be reintroduced or administered to the subject to activate, implement, and/or result in anti-tumor responses in the subject. To facilitate administration, the transduced T cells may be made into a pharmaceutical composition or made into an implant appropriate for administration in vivo, with appropriate pharmaceutically acceptable carriers or diluents. The means of making such a composition or an implant have been described in the art (see, for instance, Remington: The Science and Practice of Pharmacy, 22^{nd} edition, Pharmaceutical Press, 2012). Where appropriate, transduced T cells expressing a TCR or CAR can be formulated into a preparation in semisolid or liquid form, such as a capsule, solution, injection, in the usual ways for their respective route of administration. Means known in the art can be utilized to prevent or minimize release and absorption of the composition until it reaches the target tissue or organ, or to ensure timed-release of the composition. Generally, a pharmaceutically acceptable form is preferably employed that does not significantly adversely affect the cells expressing the TCR or chimeric receptor. In some embodiments, the transduced T cells can be made into a pharmaceutical composition containing a balanced salt solution such as Hanks' balanced salt solution, or normal saline.

The cultured T cells can be pooled and rapidly expanded. Rapid expansion provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days. In some embodiments, allogenic T cells can be pooled from several donors.

Expansion can be accomplished by a variety of methods known in the art. For example, T cells can be rapidly expanded using non-specific TCR stimulation in the presence of feeder lymphocytes and either interleukin-2 (IL-2) or interleukin-15 (IL-15), with IL-2 being preferred. The non-specific TCR stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or cells) of the cancer, which can be optionally expressed from a vector, such as an human leukocyte antigen A2 (HLA-A2) binding peptide, in the presence of a T cell growth factor, such as 300 IU/ml IL-2 or IL-15, with IL-2 being preferred. The in vitro-induced T cells are rapidly expanded by re-stimulation with the same antigen(s) of the cancer pulsed onto HLA-A2-expressing antigen-presenting cells. Alternatively, the T cells can be re-stimulated with irradiated, autologous lymphocytes or with irradiated HLA-A2+ allogeneic lymphocytes and IL-2, for example.

The autologous T cells can be modified to express a T cell growth factor that promotes the growth and activation of the autologous T cells. Suitable T cell growth factors include, for example, interleukin (IL)-2, IL-7, IL-15, and IL-12. Suitable methods of modification are known in the art including, e.g., Sambrook et al., 2001; and Ausubel et al., 1994. In some embodiments, modified autologous T cells express the T cell growth factor at high levels. T cell growth factor coding sequences, such as that of IL-12, are readily available in the art, as are promoters that can be used to promote high-level expression.

In certain embodiments, a T cell growth factor that promotes the growth and activation of the autologous or allogenic T cells is administered to the subject either concomitantly with the autologous T cells or subsequently to the autologous T cells. The T cell growth factor can be any suitable growth factor that promotes the growth and activation of the autologous T cells. Examples of suitable T cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T cell growth factor.

The T cell may be administered intravenously, intramuscularly, subcutaneously, transdermally, intraperitoneally, intrathecally, parenterally, intrathecally, intracavitary, intraventricularly, intra-arterially, via the cerebrospinal fluid, or by any implantable or semi-implantable, permanent or degradable device. The appropriate dosage of the T cell therapy may be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, a volume of about 4-10 ml (in particular 10 ml) can be administered, while for tumors of <4 cm, a volume of about 1-3 ml can be used (e.g., 3 ml). Multiple injections delivered as single dose may comprise about 0.1 to about 0.5 ml volumes.

B. Antigen-Presenting Cells

Antigen-presenting cells (APCs) are a heterogeneous group of immune cells that mediate the cellular immune response by processing and presenting antigens for recognition by certain lymphocytes such as T cells. APCs include dendritic cells, macrophages, Langerhans cells and B cells. APCs can process a protein antigen, break it into peptides, and present it in conjunction with major histocompatibility complex (MHC) molecules on the cell surface where it may interact with appropriate T cell receptors. APCs are distinguished by their expression of a particular MHC molecule. The MHC is a large genetic complex with multiple loci. The MHC loci encode two major classes of MHC membrane molecules, referred to as class I and class II MHCs. T helper lymphocytes generally recognize antigen associated with MHC class TT molecules, and T cytotoxic lymphocytes recognize antigen associated with MHC class I molecules. In humans the MHC is referred to as the HLA complex and in mice the H-2 complex.

In some embodiments, a peptide (e.g., SEQ ID NO:5) is recognized by HLA-A2 and can be used to expand antigen specific T cells in vitro. The peptide, or a nucleic acid encoding the peptide, can be used to stimulate antigen-presenting cells (APC) to trigger immune response initiation. In some embodiments, the peptide, or a corresponding polynucleotide that encodes the peptide, can be loaded onto dendritic cells, lymphoblastoid cell lines (LCL), PBMC or artificial antigen presenting cells (aAPCs), and then co-cultured with the T cells for several rounds of stimulation to generate antigen-specific CTL cell lines or clones. Expanded T cell populations that selectively recognize a Hormad1-derived peptide/HLA-A2 complex can thus be adoptively transferred to patients to treat a cancer or induce tumor regression.

In some cases, artificial antigen presenting cells (aAPCs) are useful in preparing TCR or CAR-based therapeutic compositions and cell therapy products. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009).

aAPCs may be used to expand T cells expressing a TCR or CAR. During encounter with tumor antigen, the signals delivered to T cells by antigen-presenting cells can affect T cell programming and their subsequent therapeutic efficacy. This has stimulated efforts to develop artificial antigen-presenting cells that allow optimal control over the signals provided to T cells (Turtle et al., 2010). In addition to antibody or antigen of interest, the aAPC systems may also comprise at least one exogenous assisting molecule. Any suitable number and combination of assisting molecules may be employed. The assisting molecule may be a co-stimulatory molecule or an adhesion molecule. Exemplary co-stimulatory molecules include CD70 and B7.1 (also called B7 or CD80), which can bind to CD28 and/or CTLA-4 molecules on the surface of T cells, thereby promoting, e.g., T cell expansion, Th1 differentiation, short-term T cell survival, and cytokine secretion such as interleukin (IL)-2 (see Kim et al., 2004). Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs) that promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001. C. Nucleic Acids

In an aspect, the present disclosure provides a nucleic acid encoding an isolated TCR (e.g., sTCR), CAR, or peptide as disclosed herein. For example the nucleic acid may encode a polypeptide comprising a TCR variable region having about 90%, 91%, 92%, 93%, 94%, 95%, 96%, 97%, 98%, 99%, or 100% sequence identity to a TCR variable region disclosed herein (e.g., SEQ ID NO: 1-4), or a TCR variable region having 1, 2, 3, or 4 point mutations (e.g., substitution mutations) as compared to any one of SEQ ID NO: 1-4. The term “nucleic acid” is intended to include DNA and RNA and can be either double stranded or single stranded.

Accordingly, a nucleic acid encoding a TCR (e.g., sTCR), CAR, or peptide may be operably linked to a promoter and/or comprised in an expression vector. The TCR, CAR or peptide can be produced in the appropriate expression system using methods well known in the molecular biological arts. A nucleic acid encoding a tumor antigen-specific peptide disclosed herein may be incorporated into any expression vector which ensures good expression of the peptide in the desired environment (e.g., in human immune cells). Possible vectors that can be used include but are not limited to cosmids, plasmids, or modified viruses (e.g. replication defective retroviruses, adenoviruses and adeno-associated viruses), so long as the vector is suitable for transformation of a host cell.

A recombinant expression vector being “suitable for transformation of a host cell” means that the expression vector contains a nucleic acid molecule of the present disclosure and regulatory sequences selected on the basis of the host cells to be used for expression, which are operatively linked to the nucleic acid molecule. The terms, “operatively linked” or “operably linked” are used interchangeably, and are intended to mean that the nucleic acid is linked to regulatory sequences in a manner which allows expression of the nucleic acid under the control of those regulatory sequences.

Accordingly, the present invention provides a recombinant expression vector comprising nucleic acid encoding a TCR, CAR, or soluble peptide that selectively binds Hormad1, and the necessary regulatory sequences for the transcription and translation of the inserted protein-sequence. Suitable regulatory sequences may be derived from a variety of sources, including bacterial, fungal, or viral genes (e.g., see the regulatory sequences described in Goeddel, 1990).

Selection of appropriate regulatory sequences is generally dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions. Indeed, in some embodiments, it is preferable to employ a native regulatory sequence (e.g., a promoter) associated with expression of the TCR in the organism from which it was obtained.

A recombinant expression vector may also contain a selectable marker gene which facilitates the selection of host cells transformed or transfected with the TCR, CAR, or soluble peptide that selectively binds Hormad1 disclosed herein. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformed cells can be selected with G418 (Geneticin); thus, cells that have incorporated the selectable marker gene will survive, while the other cells die when exposed to the antibiotic. This makes it possible to visualize and assay for expression of a recombinant expression vector, and also to determine the effect of a mutation on expression and phenotype.

Recombinant expression vectors can be introduced into host cells to produce a transformed host cell. The term “transformed host cell” is intended to include prokaryotic and eukaryotic cells which have been transformed or transfected with a recombinant expression vector of the invention. The terms “transformed with”, “transfected with”, “transformation”, and “transfection” are intended to encompass introduction of nucleic acid (e.g. a vector) into a cell by one of many possible techniques known in the art. Suitable host cells include a wide variety of prokaryotic and eukaryotic host cells. For example, the proteins of the present disclosure may be expressed in bacterial cells such as E. coli, insect cells (using baculovirus), yeast cells, or mammalian cells.

A nucleic acid molecule of the present disclosure may also be chemically synthesized using standard techniques. Various methods of chemically synthesizing polydeoxy-nucleotides are known, including solid-phase synthesis which, like peptide synthesis, has been automated in commercially available DNA synthesizers (See e.g., U.S. Pat. Nos. 4,598,049; 4,458,066; 4,401,796; and 4,373,071).

### III. PEPTIDE VACCINES

In some aspects, methods are provided for the treatment of a cancer (e.g., a breast cancer, a lung cancer, etc.) comprising immunizing a subject with a purified tumor antigen or an immunodominant tumor antigen-specific peptide such as a Hormad1 peptide (SEQ ID NO:5). The Hormad1 peptide can be administered to a mammalian subject, such as a human patient, via a variety of routes (e.g., intramuscular, intravenous, subcutaneous, etc.). In some embodiments, the peptide can be injected in a solution (e.g., a saline solution) as a vaccine or to cause an immune response against the peptide. For example, in order to enhance the solubility of the peptide and/or increase the immune response in the subject, an adjuvant can be included in the formulation or solution (e.g., as described in Massarelli et al., 2019). Peptide pulsed mature dendritic cells can be administered to the subject in some embodiments. Approaches that may be used to cause an immune response or anti-cancer response against the peptide in a subject include, e.g., those described in Wen et al. (2019) and Massarelli et al. (2019). In some embodiments, the Hormad1 peptide (SEQ ID NO:5) is bound to or presented by autologous dendritic cells that can be reinfused to a subject or human patient.

### IV. ANTICANCER THERAPIES

Embodiments of the disclosure relate to the administration of additional anticancer therapies. In some embodiments, the additional anticancer therapy is one described herein. Examples of additional anticancer therapies are provided below.

A. Immunostimulators

In some embodiments, the method further comprises administration of an additional agent. In some embodiments, the additional agent is an immunostimulator. The term “immunostimulator” as used herein refers to a compound that can stimulate an immune response in a subject, and may include an adjuvant. In some embodiments, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL® (ASO4), MPL A of above-mentioned bacteria separately, saponins, such as QS-21, Quil-A, ISCOMs, ISCOMATRIX, emulsions such as MF59, Montanide, ISA 51 and ISA 720, ASO2 (QS21+squalene+MPL), liposomes and liposomal formulations such as ASO1, synthesized or specifically prepared microparticles and microcarriers such as bacteria-derived outer membrane vesicles (OMV) of N. gonorrhoeae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, the additional agent comprises an agonist for pattern recognition receptors (PRR), including, but not limited to Toll-Like Receptors (TLRs), specifically TLRs 2, 3, 4, 5, 7, 8, 9 and/or combinations thereof. In some embodiments, additional agents comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some embodiments, the additional agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an additional agent may be a TLR-4 agonist, such as bacterial lipopolysaccharide (LPS), VSV-G, and/or HMGB-1. In some embodiments, additional agents may comprise TLR-5 agonists, such as flagellin, or portions or derivatives thereof, including but not limited to those disclosed in U.S. Pat. Nos. 6,130,082, 6,585,980, and 7,192,725.

In some embodiments, additional agents may be proinflammatory stimuli released from necrotic cells (e.g., urate crystals). In some embodiments, additional agents may be activated components of the complement cascade (e.g., CD21, CD35, etc.). In some embodiments, additional agents may be activated components of immune complexes. Additional agents also include complement receptor agonists, such as a molecule that binds to CD21 or CD35. In some embodiments, the complement receptor agonist induces endogenous complement opsonization of the synthetic nanocarrier. In some embodiments, immunostimulators are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

B. Immunotherapies

In some embodiments, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

1. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and TFNβ), type TT (IFNγ) and type III (IFNλ).

Interleukins have an array of immune system effects. IL-2 is an exemplary interleukin cytokine therapy.

5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

6. Checkpoint Inhibitors and Combination Treatment

In some embodiments, the additional therapy comprises immune checkpoint inhibitors. Certain embodiments are further described below.

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

C. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

D. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

E. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

F. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

G. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

H. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

I. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. PROTEINACEOUS COMPOSITIONS

As used herein, a “protein” “peptide” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a peptide, protein, or polypeptide (wild-type or modified), such as a peptide or protein of the disclosure comprising a peptide of SEQ ID NO:5, or the TCR embodiments of SEQ ID NOS:2, 4, 6-11, 13, or 15 may comprise, but is not limited to at least, at most, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include at least, at most, exactly, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous in sequence to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 contiguous amino acids or nucleic acids of SEQ ID NO:1-15. In certain embodiments, the peptide or polypeptide is not naturally occurring and/or is in a combination of peptides or polypeptides.

In some embodiments, the protein or polypeptide or nucleic acid may comprise amino acids or nucleic acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 (or any derivable range therein) of one of SEQ ID NO:1-15. In some embodiments, the peptides of the disclosure comprise at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) flanking the caboxy and/or flanking the amino end of a peptide comprising or consisting of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222,223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260,261, 262, 263, 264, 265, 266, 267, 268, 269, or 270 contiguous amino acids of one of SEQ ID NO: 2, 4, 5-11, 13, or 15.

In some embodiments, the protein, polypeptide, or nucleic acid may comprise at least, at most, exactly, or about 1, 2, 3, 44, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, or 270 (or any derivable range therein) contiguous amino acids of one of SEQ ID NO: 2, 4, 5-11, 13, or 15.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, or 20 (or any derivable range therein) contiguous amino acids of a peptide or nucleic acid of SEQ ID NO:1-15 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NO:1-15.

In some aspects there is a polypeptide, nucleic acid (or a nucleic acid molecule encoding such a polypeptide) starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, or 270 of one of SEQ ID NO:1-15 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, or 270 (or any derivable range therein) contiguous amino acids of one of SEQ ID NO:1-15.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of a polypeptide with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VI. PHARMACEUTICAL PREPARATIONS

In select embodiments, it is contemplated that a Hormad1-derived peptide (e.g., SEQ ID NO:5), a cell (e.g., a T cell) expressing a TCR as disclosed herein (e.g., any of SEQ ID NOs: 1-4), or a protein containing the variable regions of a TCR of the present disclosure may be administered to a subject to induce a therapeutic immune response in the subject towards a cancer (e.g., a solid tumor that expresses Hormad1). A pharmaceutical composition for use in a subject may comprise a TCR disclosed herein, such as a soluble TCR (optionally attached to an imaging agent or a therapeutic agent) or a bispecific TCR, and a pharmaceutically acceptable carrier. If desired, the pharmaceutical composition may contain an additional immunostimulatory compound or anti-cancer agent.

The phrases “pharmaceutical,” “pharmaceutically acceptable,” or “pharmacologically acceptable” refers to molecular entities and compositions that do not produce an adverse, allergic or other untoward reaction when administered to an animal, such as, for example, a human, as appropriate. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington: The Science and Practice of Pharmacy, 22^{nd} edition, Pharmaceutical Press, 2012, incorporated herein by reference). Except insofar as any conventional carrier is incompatible with the proteins (e.g., a Hormad1 peptide, a soluble TCR) or cells (e.g., a T cell expressing a TCR) of the present disclosure, its use in the vaccine compositions or adoptive cell transfer therapies of the present invention is contemplated.

As used herein, a “therapeutic immune response” or a “protective immune response” refer to a response by the immune system of a mammalian host to a cancer. A protective immune response may provide a therapeutic effect for the treatment of a cancer, e.g., decreasing tumor size, increasing survival, etc.

A person having ordinary skill in the medical arts will appreciate that the actual dosage amount of a therapeutic composition administered to an animal or human patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

A therapeutic composition disclosed herein can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, intraperitoneally, subcutaneously, subconjunctivally, intravesicularlly, mucosally, intrapericardially, intraocularly, orally, topically, locally, or by injection, infusion, continuous infusion, lavage, and localized perfusion. A therapeutic composition may also be administered to a subject via a catheter, in lipid compositions, or by other method or any combination of the forgoing as would be known to one of ordinary skill in the art (see, for example, Remington: The Science and Practice of Pharmacy, 22^{nd} Ed., Pharmaceutical press, 2012, incorporated herein by reference).

While any suitable carrier known to those of ordinary skill in the art may be employed in the pharmaceutical compositions of this invention, the type of carrier will vary depending on the mode of administration. For parenteral administration, such as intravenous, intratumoral or subcutaneous injection, the carrier may comprise water, saline, alcohol, a fat, a wax or a buffer. Biodegradable microspheres (e.g., polylactic galactide) may also be employed as carriers in some embodiments. Suitable biodegradable microspheres are disclosed, for example, in U.S. Pat. Nos. 4,897,268 and 5,075,109.

In some embodiments, the vaccine composition may be administered by microstructured transdermal or ballistic particulate delivery. Microstructures as carriers for vaccine formulation are a desirable configuration for vaccine applications and are widely known in the art (e.g., U.S. Pat. Nos. 5,797,898, 5,770,219 and 5,783,208, and U.S. Patent Application 2005/0065463). Microstructures or ballistic particles that serve as a support substrate for an TCR, such as a soluble TCR, disclosed herein may be comprised of biodegradable material and non-biodegradable material, and such support substrates may be comprised of synthetic polymers, silica, lipids, carbohydrates, proteins, lectins, ionic agents, crosslinkers, and other microstructure components available in the art. Protocols and reagents for the immobilization of a peptide of the invention to a support substrate composed of such materials are widely available commercially.

In other embodiments, a vaccine composition comprises an immobilized or encapsulated TCR or soluble TCR disclosed herein and a support substrate. The support substrate can include, but is not limited to, a lipid microsphere, a lipid nanoparticle, an ethosome, a liposome, a niosome, a phospholipid, a sphingosome, a surfactant, a transferosome, an emulsion, or a combination thereof. The formation and use of liposomes and other lipid nano- and microcarrier formulations is generally known to those of ordinary skill in the art, and the use of liposomes, microparticles, nanocapsules and the like have gained widespread use in delivery of therapeutics (e.g., U.S. Pat. No. 5,741,516, specifically incorporated herein in its entirety by reference). Numerous methods of liposome and liposome-like preparations as potential drug carriers, including encapsulation of peptides, are known and may be used in various embodiments (e.g., U.S. Pat. Nos. 5,567,434, 5,552,157, 5,565,213, 5,738,868, and 5,795,587).

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

A. Combination Therapies

In certain embodiments, the compositions and methods of the present embodiments comprising an antigen-specific cell (e.g., autologous or allogeneic T cells (e.g., regulatory T cells, CD4+ T cells, CD8+ T cells, α-β T cells, or γ-δ T cells), NK cells, invariant NK cells, NKT cells, mesenchymal stem cell (MSC)s, or induced pluripotent stem (iPS) cells) population may be administered to a mammalian subject (e.g., a human) in combination with at least one additional therapy. The additional therapy may be radiation therapy, surgery (e.g., a primary surgery, a tumor removal, a lumpectomy, or a mastectomy), chemotherapy, a conditioning chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of a small molecule enzymatic inhibitor or anti-metastatic agent. In some embodiments, the additional therapy is the administration of one or more side-effect limiting agents (e.g., agents that may lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is a chemotherapy such as, e.g., dacarbazine, or temozolomide. The additional therapy may be one or more of the chemotherapeutic agents known in the art.

A T cell therapy or adoptive cell transfer therapy may be administered before, during, after, or in various combinations relative to an additional cancer therapy, such as immune checkpoint therapy or conditioning chemotherapy. The administrations may be in intervals ranging from concurrently to minutes to days to weeks. In embodiments where the T cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 hours of each other and, more particularly, within about 6-12 hours of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an antigen-specific T cell therapy, peptide, or TCR is “A” and an anti-cancer therapy is “B”: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Hormad1-Specific T Cell Receptor Redirects T Cells Against Tumor Cells

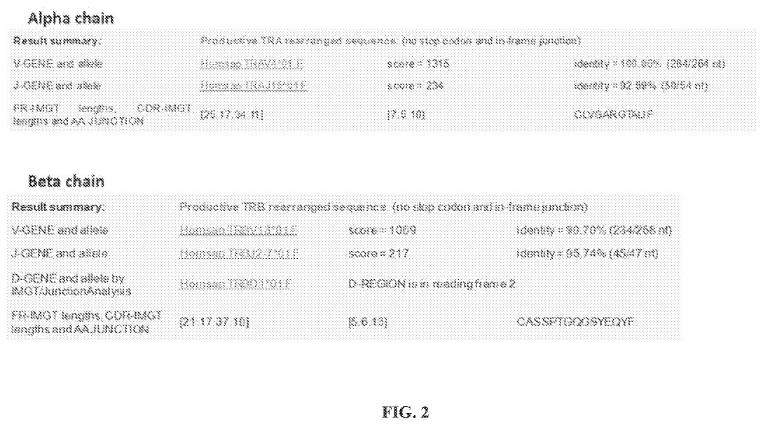

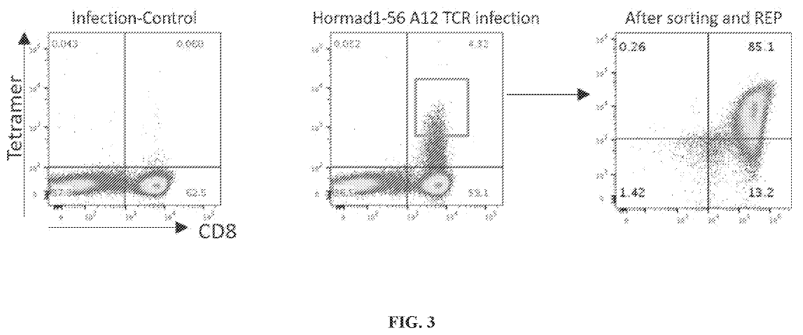

To further explore the potential of Hormad1 T cell epitope as a therapeutic target for clinical immunotherapy, the whole length Hormad1-56 TCR α chain and β chain were inserted into retrovirus vector pMSGV3 and then the recombinant retrovirus vector was used to infect the PBMCs (FIG. 3). The empty retrovirus vector was used as a control. After infection, the CD8+/Tetramer+ population was observed with FCM detection. After tetramer guided sorting and expansion, a high purity of TCR-T cells was generated. While Hormad1 over-expression was observed in tumor tissues from about 50% of non-small cell lung cancer (NSCLC) patients tested, elevated Hormad1 expression was not observed in healthy tissues (Hormad1 is not expressed in healthy tissues other than in the testis), and high Hormad1 expression was correlated with elevated mutation burden in the lung adenocarcinoma patient population (Nichols et al., 2018), it was unclear if this protein could be used as a target for immunotherapies.

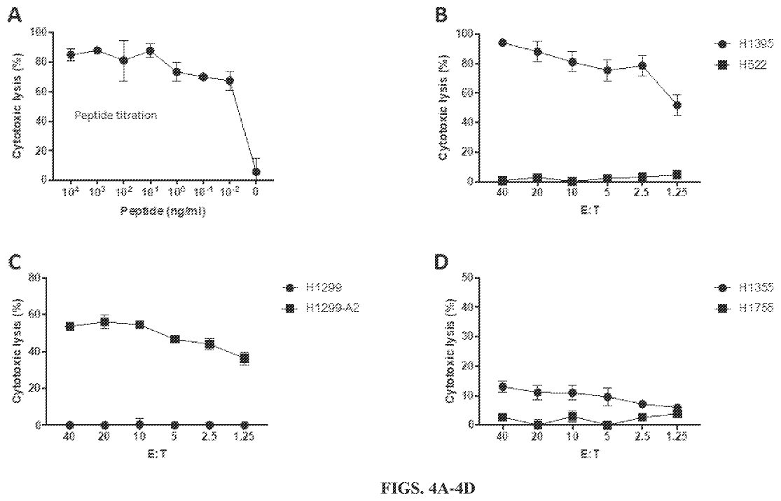

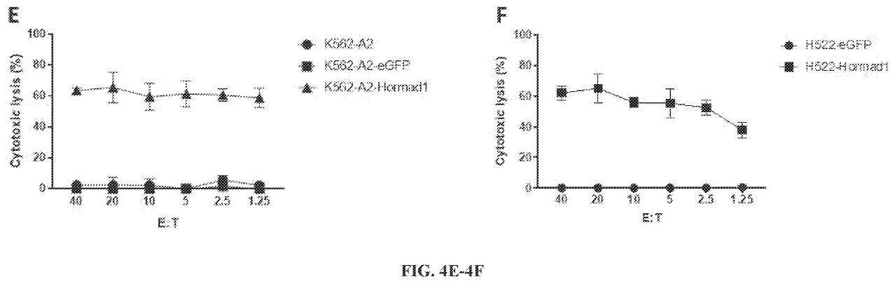

It was observed that the TCR-transduced T cells specifically recognized Hormad1 peptide-pulsed T2 cells at high avidity and lyse the HLA-A2+, Hormad1-expressing tumor cell lines, but not Hormad1-, or HLA-A2-, or normal cells (FIG. 4). Hormad1-specific TCR-transduced T cells can recognize these solid tumor cells but not control tumor cells (FIG. 4). These results suggest that Hormad1-derived peptides are expressed in the context of HLA-A2 molecules on tumor cells and Hormad1-TCR transduced T cells can be used for immunotherapy cancers.

### Hormad1-56 TCR-T Functional Assay

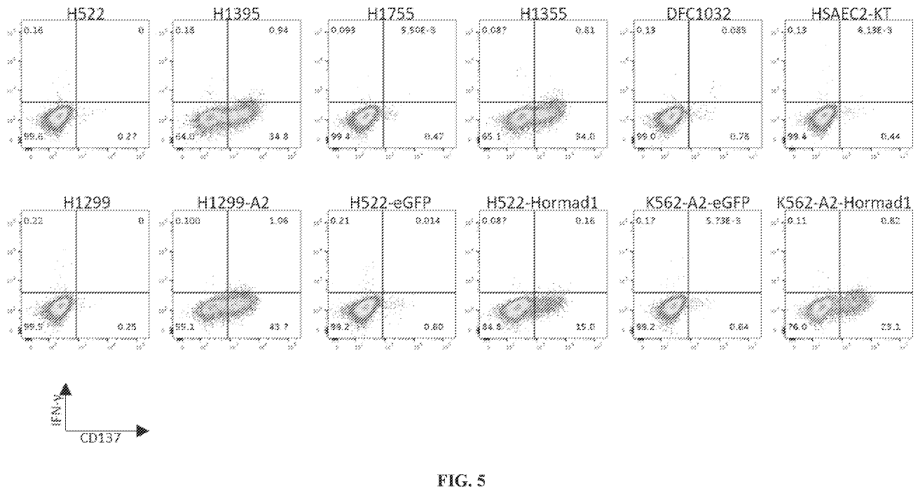

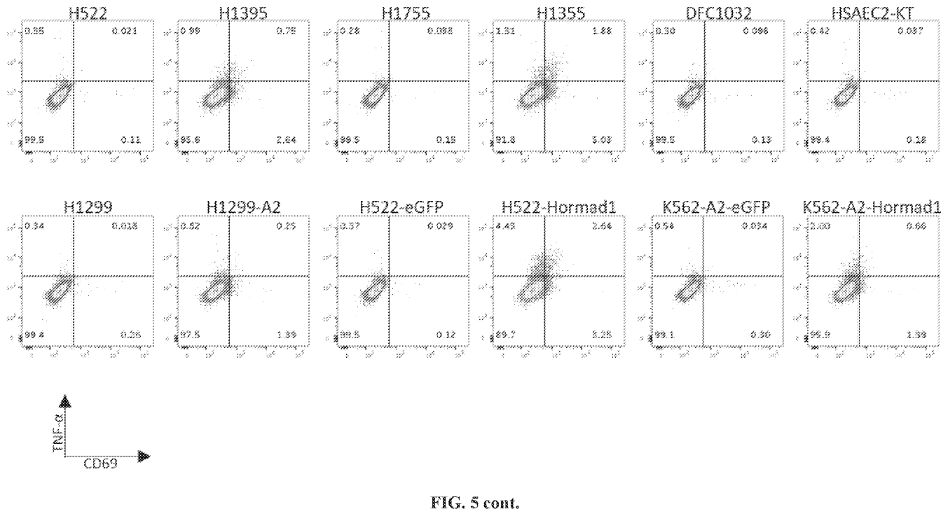

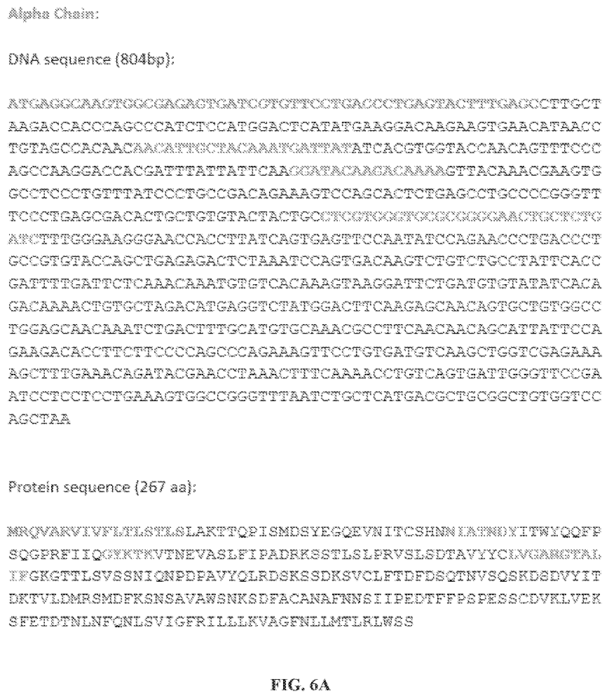

In order to further explore the Hormad1-56 TCR-T function, cytokine production was detected by intracellular staining assay (Pala Pietro, et al., J Immunol Methods. 2010; 243(1-2):107-124). Hormad1-56 TCR-T were co-cultured with several tumor cell lines. It was observed that the levels of CD137, CD69, TNF-α, IFN-γ expressed by TCR-T were enhanced significantly when co-cultured with HLA-A2+, Hormad1-expressing tumor cell lines, but not antigen-negative cells, as well as control tumor cell lines (FIG. 5).

The TCR sequence was derived from parental Hormad1-56 CTL cell line A12. It revealed the Hormad1-56 TCRs (hereafter referred to as Hormad1-TCR) were composed of TRAV4*01 F, TRBV13*01 F and TRAV4*01 F, TRBV13*01 F 2 subfamily sequences (FIG. 6).

### Example 2

### Materials and Methods

### Healthy Donor PBMC Samples

The Institutional Review Board of The University of Texas M. D. Anderson Cancer Center approved the study. An informed consent was obtained in accordance with the Declaration of Helsinki prior to collection of healthy donor PBMC samples. Peripheral blood mononuclear cells (PBMC) were isolated from blood samples by leukapheresis.

### Cell Lines

T2 hybridoma cells, lung cancer cell lines H1395, H522, H1299, H1299-A2, H1355, H1755, DFC1032, K562-A2, K562-A2-eGFP, K562-A2-Hormad1, H522-eGFP, H522-Hormad1, were cultured in RPMI 1640 medium supplemented with 10% fetal bovine serum, 10 mM HEPES, 1× Glutamax, 50 μM β-mercaptoethanol, 1 mM sodium pyruvate, 100 U/mL penicillin+100 μg/mL streptomycin, and 10 μg/mL gentamicin (all from Invitrogen, Carlsbad, Calif.) at 37° C. and 5% CO₂ in air. The normal lung cell line HSAEC2-KT was cultured in serum free Small Airway Epithelial Cell Growth Medium (PromoCell, Heidelberg, Germany).

### Tumor and Immune Cell Subset Isolation

CD25− T cells were isolated by magnetic cell separation (MACS, Miltenyi Biotec, Auburn, Calif.) and the purity confirmed by flow. The procedure yielded >90% purity of CD25− T cells.

### Reagents

Mouse anti-human antibodies against CD3, CD4, CD8, CD69, CD137, IFN-γ, TFN-α were obtained from Biolegend, San Diego, Calif. All peptides were synthesized by Genscript, Piscataway, N.J. to greater than 90% purity and dissolved in dimethyl sulfoxide (Sigma-Aldrich). PE-conjugated tetramers were synthesized by Immune Monitoring Center of Fred Hutchinson Cancer Research Center, Seattle, Wash.

### PCR

Total RNA was extracted from T cells using RNeasy Kit (Qiagen). About 3 μg of total RNA was reverse transcribed into cDNA with SMARTer® RACE 5′/3′ Kit (ClonTech). The TCR fragment cloning with PCR was performed with Q5® High-Fidelity 2× Master Mix kit (NEB) using the following conditions: 98° C. for 2 min, followed by 98° C. for 15 sec, 63° C. for 30 sec, 72° C. for 45 sec for 40 cycles on Bio-Rad PCR System. The PCR products were cloned into pRACE vector with In Fusion clone kit (ClonTech) and then for DNA sequencing with BigDye® Direct Cycle Sequencing Kits (Thermo)

### Flow Cytometry

For intracellular staining, cells were fixed and permeabilized using Fixation/Permeabilization kit (eBioscience) as per manufacturer's instructions. Cells were then stained with mouse anti-human-flow antibody (as described above; 00114) for 30 min at 4° C. After two washes, samples were acquired on a FACS Calibur (BD Biosciences) and analyzed using Cell Quest Pro (BD Biosciences) or FlowJo (Tree Star, Inc., Ashland, Oreg.) software. Intracellular cytokine staining was performed as previously described (Weng et al., 2016b). For tetramer staining, PE-conjugated Hormad1 tetramer and APC-Cy7-conjugated mouse anti-human CD8 antibody were mixed with the cells in 50 μl volume for 30 minutes at room temperature, washed twice, and analyzed by a flow cytometry.

### Hormad1-56 Peptide Specific CTL Line Generation

The mature DC derived from HLA-A0201+ healthy donor were pulsed with Hormad1-56 peptide (YLDDLCVKI; SEQ ID NO:5) and stimulated autologous CD25− T cells. After two rounds of stimulation, Hormad1-56 specific T cell lines were detected and sorted with corresponding Hormad1-56 tetramer and anti-CD8 antibody. The CD8+/Tetramer+ T cells were expanded with rapid expansion protocol (REP) and the purity of Hormad1-56 specific T cells was determined with anti-CD8 antibody and Tetramer staining.

### Generation of Hormad1-Specific TCR-T Cells by Retrovirus

The full TCRαp sequences of Hormad1 T cell line was obtained by 5-RACE RT-PCR and codon-optimized. The constant regions of α and β chains were cysteine-mutated; the TCRαp chains were ligated with Furin and P2A and cloned into a retrovirus producing vector. TCR-containing retrovirus were produced in 293T cells, filtered, concentrated and stored at −80° C. HLA-A2+ healthy donors T cells were activated by OKT3 antibody and IL-2 for 72 hours and the transduction with retrovirus was carried out at 2000 g centrifuge at 32° C. for 2 hours followed by overnight incubation. The expression of antigen specific TCR was analyzed by tetramer-staining 48 hours later. The tetramer positive T cells were sorted by flow and further expanded by REP for additional functional assays as previously described (Pollack et al., 2014).

### Cytotoxicity Assay

T2 cells were pulsed with decreasing concentrations of peptide (10 μg/ml to 10 pg/ml) and used as targets in standard 4-hour Cr51 release cytotoxicity assay. Tumor cell line (2×10³ cells/well) were incubated with the effector T cells at the indicated ratios (From E:T=40:1 to 1.25:1) in 96-well round-bottom plates at 37° C. for 4 hours, and target cell lysis was determined by Cr51 release assay. All assays were performed in triplicate wells and repeated at least two times.

### Statistical Analysis

The Student t test was used to compare various experimental groups. P values <0.05 were considered statistically significant. Unless otherwise indicated, mean and standard deviations are shown.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,373,071
U.S. Pat. No. 4,458,066
U.S. Pat. No. 4,598,049
U.S. Pat. No. 4,897,268
U.S. Pat. No. 5,075,109
U.S. Pat. No. 5,552,157
U.S. Pat. No. 5,565,213
U.S. Pat. No. 5,567,434
U.S. Pat. No. 5,738,868
U.S. Pat. No. 5,741,516
U.S. Pat. No. 5,770,219
U.S. Pat. No. 5,783,208
U.S. Pat. No. 5,795,587
U.S. Pat. No. 5,797,898
U.S. Pat. No. 6,225,042
U.S. Pat. No. 6,355,479
U.S. Pat. No. 6,362,001
U.S. Pat. No. 6,790,662
U.S. Pat. No. 6,410,319
U.S. Pat. No. 7,666,604
U.S. Patent Appl. No. 2009/0017000
U.S. Patent Appl. No. 2009/0004142
U.S. Patent Appl. No. 2005/0065463
WO 2007/103009
WO 99/60120
Ausubel et al., Current Protocols in Molecular Biology, Greene Publishing Associates and John Wiley & Sons, N Y, 1994.
Boulter et al., Clin Exp Immunol.; 142(3): 454-460, December 2005.
Chothia et al., EMBO J. 7:3745, 1988.
Cohen et al., Enhanced Antitumor Activity of T Cells Engineered to Express T cell Receptors
with a Second Disulfide Bond. Cancer research; 67(8):3898-903, 2007.
Dileepan et al., PLoS One.; 10(6): Jun. 11, 2015.
Dossinger et al. PLoS One.; 8(4), Apr. 26, 2013.
Eshhar et al., Methods, vol 8(2):133-142, 1995.
Goeddel, Methods Enzymol., 185:3-7, 1990.
Heemskerk et al. Hum Gene Ther. 19:496-510, 2008.
Hiwarkar et al., Blood, 126 (26): 2882-2891, 2015.
Johnson et al., Gene therapy with human and mouse T cell receptors mediates cancer regression and targets normal tissues expressing cognate antigen. Blood; 114(3):535-46. 2009.
Jores et al., PNAS U.S.A. 87:9138, 1990.
Kabat et al., “Sequences of Proteins of Immunological Interest, US Dept. Health and Human Services, Public Health Service National Institutes of Health, 5th ed, 1991.
Kim et al., Nature, 22(4):403-410, 2004.
Kochenderfer et al., Chemotherapy-refractory diffuse large B-cell lymphoma and indolent B-cell malignancies can be effectively treated with autologous T cells expressing an anti-CD19 chimeric antigen receptor. J Clin Oncol; 33(6):540-9, 2015.
Lefranc et al., Dev. Comp. Immunol. 27:55, 2003.
Levine et al., Mol Ther Methods Clin Dev., 4: 92-101, 2017.
Locke et al., Phase 1 Results of ZUMA-1: A Multicenter Study of KTE-C19 Anti-CD19 CAR T Cell Therapy in Refractory Aggressive Lymphoma. Mol Ther.; 25(1):285-95, 2017.
Massarelli E, et al. JAMA Oncol. 2019 Jan. 1; 5(1):67-73.
McCromack et al. Cancer Immunol Immunother.; 62(4):773-85, April 20-13.
Neal et al., J Immunol Res Ther.; 2(1): 68-79, 2017.
Neelapu et al., Axicabtagene ciloleucel CAR T cell therapy in refractory large B-cell lymphoma. N Engl J Med.; 377(26):2531-2544, Dec. 28, 2017.
Nichols et al., Cancer Res.; 78(21):6196-6208, 2018.
Oates et al. Oncoimmunology.; 2(2), Feb. 1, 2013:
Plosker and Figgitt, Rituximab: a review of its use in non-Hodgkin's lymphoma and chronic lymphocytic leukaemia. Drugs; 63(8):803-43, 2003.
Pollack S M et al. J Immunother Cancer.; 2(1):36, 2014.
Porter et al., Chimeric antigen receptor-modified T cells in chronic lymphoid leukemia. The New England journal of medicine; 365(8):725-33, 2011.
Remington: The Science and Practice of Pharmacy, 22^{nd} Ed., Pharmaceutical Press, 2012.
Riet et al., Methods Mol Biol.; 969:187-201, 2013.
Sambrook et al., Molecular Cloning: A Laboratory Manual, 3rd ed., Cold Spring Harbor Press, Cold Spring Harbor, N.Y., 2001.
Schamel et al. J Exp Med.; 202(4): 493-503, Aug. 15, 2005.
Schuster et al., Sustained Remissions Following Chimeric Antigen Receptor Modified T Cells Directed Against CD19 (CTL019) in Patients with Relapsed or Refractory CD19+ Lymphomas. Blood; 126(23):183-, 2015.
Sotillo et al., Convergence of Acquired Mutations and Alternative Splicing of CD19 Enables Resistance to CART-19 Immunotherapy. Cancer Discov; 5(12):1282-95, 2015.
Strome et al., Cancer Res.; 62(6):1884-9, Mar. 15, 2002.
Terakura et al., Blood. 1:72-82, 2012.
Topp et al., Phase II trial of the anti-CD19 bispecific T cell-engager blinatumomab shows hematologic and molecular remissions in patients with relapsed or refractory B-precursor acute lymphoblastic leukemia. J Clin Oncol.; 32(36):4134-40, 2014.
Turtle et al., “Artificial antigen-presenting cells for use in adoptive immunotherapy” Cancer J.;16(4):374-81, July-August 2010.
Turtle et al., CD19 CAR-T cells are highly effective in ibrutinib-refractory chronic lymphocytic leukemia. Blood.; 128(56), 2016b.
Walseng et al., A TCR-based Chimeric Antigen Receptor. Scientific Reports; 7(1):10713, 2017.
Walseng et al., PLoS One.; 10(4), Apr. 13, 2015.
Wang et al., J Immunother. 35(9):689-701, 2012.
Wen P Y, et al. Clin Cancer Res. (19):5799-5807, 2019.
Weng et al., IL-15 enhances the antitumor effect of human antigen-specific CD8+ T cells by cellular senescence delay. Oncoimmunology; 5(12), 2016b.
Zhang et al., BMC Biotechnol., 18:4, 2018.

1. An isolated Hormad1 peptide of 35 amino acids in length or less comprising:
i) SEQ ID NO:5,
ii) an amino acid sequence with at least 85% sequence identity to SEQ ID NO:5,
iii) an amino acid sequence comprising at least 6 contiguous amino acids of SEQ ID NO:5, or
iv) an amino acid sequence that has only one substitution mutation relative to SEQ ID NO:5.

2. The peptide of claim 1, wherein the peptide is 30 amino acids in length or less.

3. The peptide of claim 2, wherein the peptide is 15 amino acids in length or less.

4. The peptide of claim 3, wherein the peptide is 10 amino acids in length or less.

5. The peptide of claim 1, wherein the peptide consists of SEQ ID NO: 5.

6. The peptide of any one of claims 1-5, wherein the peptide is immunogenic and/or wherein the peptide is capable of inducing cytotoxic T lymphocytes (CTLs) and selectively binds to HLA-A2.

7. The peptide of any one of claims 1-6, wherein the peptide is modified.

8. The peptide of claim 7, wherein the modification comprises conjugation to a molecule.

9. The peptide of claim 7 or 8, wherein the molecule comprises an antibody, a lipid, an adjuvant, or a detection moiety.

10. A pharmaceutical composition comprising the isolated peptide of any one of claims 1-9 and a pharmaceutical carrier.

11. The composition of claim 10, wherein the pharmaceutical composition is formulated for parenteral administration, intravenous injection, intramuscular injection, or subcutaneous injection.

12. The composition of claim 10 or 11, wherein the pharmaceutical composition comprises a liposome, lipid-containing nanoparticle, or a lipid-based carrier.

13. The composition of any one of claims 10-12, wherein the pharmaceutical preparation is formulated for injection.

14. The composition of any one of claims 10-12, wherein the pharmaceutical preparation is formulated for inhalation.

15. The composition of claim 14, wherein the pharmaceutical preparation comprises or consists of a nasal spray.

16. An isolated nucleic acid encoding the Hormad1-derived peptide of any one of claims 1-9.

17. A vector comprising the nucleic acid of claim 16.

18. An isolated host cell comprising the nucleic acid of claim 16 or the vector of claim 17.

19. A method of making a cell comprising transferring the nucleic acid of claim 16 or the vector of claim 17 into the cell.

20. A method of stimulating an immune response in a mammalian subject, comprising administering an effective amount of the peptide of any one of claims 1-9 to the subject.

21. The method of claim 20, wherein the subject has a cancer.

22. The method of claim 20 or 21, wherein the cancer is a breast cancer, a lung cancer, bone cancer, endometrial cancer, hematopoietic or lymphoid cancer, gastrointestinal cancer, ovarian cancer, skin cancer, neuroblastoma, testicular cancer, thymoma, bladder cancer, uterine carcinoma, melanoma, sarcoma, cervix cancer, or head and neck cancer.

23. The method of any one of claims 20-22, wherein the cancer comprises a cancer that is positive for expression of the peptide.

24. The method of any one of claims 20-23, wherein the method further comprises administering autologous dendritic cells to the subject, wherein the peptide is bound to or presented by the autologous dendritic cells.

25. The method of any one of claims 20-23, wherein the peptide and artificial antigen presenting cells (aAPCs) are administered to the subject, wherein the peptide is bound to or presented by the aAPCs.

26. The method of claim 25, wherein the peptide is operatively linked to the artificial antigen presenting cells (aAPCs).

27. The method of claim 26, wherein the peptide is linked through a peptide bond or through van der Waals forces.

28. The method of any one of claims 20-27, wherein the subject is a human.

29. The method of any one of claims 20-28, wherein the peptide induces, activates, or stimulates the proliferation of Hormad1-specific T cells in the subject

30. The method of any one of claims 20-29, further comprising administering at least a second anti-cancer therapy.

31. The method of claim 30, wherein the second anti-cancer therapy is selected from the group consisting of a chemotherapy, a radiotherapy, an immunotherapy, or a surgery.

32. A method of activating or expanding Hormad1-specific T cells comprising:
(a) obtaining a starting population of cells from a mammalian subject and preferably from a blood sample from the mammalian subject, wherein the starting population of cells comprises T cells; and
(b) contacting the starting population of cells ex vivo with the Hormad1-derived peptide of any one of claims 1-9, thereby activating, stimulating proliferation, and/or expanding Hormad1-specific T cells in the starting population.

33. The method of claim 32, wherein contacting is further defined as co-culturing the starting population of T cells with antigen presenting cells (APCs), wherein the APCs can present the Hormad1-derived peptide of claim 1 on their surface.

34. The method of claim 33, wherein the APCs are dendritic cells.

35. The method of claim 34, wherein the dendritic cells are autologous dendritic cells obtained from the mammalian subject.

36. The method of claim 32, wherein contacting is further defined as co-culturing the starting population of T cells with artificial antigen presenting cells (aAPCs).

37. The method of claim 36, wherein the artificial antigen presenting cells (aAPCs) comprise or consist of poly(lactide-co-glycolide) (PLGA), K562 cells, paramagnetic beads coated with CD3 and CD28 agonist antibodies, beads or microparticles coupled with an HILA-dimer and anti-CD28, or nanosize-aAPCs (nano-aAPC) that are preferably less than 100 nm in diameter.

38. The method of any one of claims 32-37, wherein the T cells are CD8⁺ T cells or CD4⁺ T cells.

39. The method of any one of claims 32-38, wherein the T cells are cytotoxic T lymphocytes (CTLs).

40. The method of any one of claims 32-39, wherein the starting population of cells comprises or consists of peripheral blood mononuclear cells (PBMCs).

41. The method of claim 40, wherein the method further comprises isolating or purifying the T cells from the peripheral blood mononuclear cells (PBMCs).

42. The method of any one of claims 32-41, wherein the mammalian subject is a human.

43. The method of any one of claims 32-42, wherein the method further comprises reinfusing or administering the activated or expanded Hormad1-specific T cells to the subject.

44. A Hormad1-specific T cell activated or expanded according to any one of claims 32-43.

45. A pharmaceutical composition comprising the Hormad1-specific T cells activated or expanded according to any one of claims 32-43.

46. An engineered T cell receptor (TCR) having antigenic specificity for Hormad1 or SEQ ID NO: 5, wherein the TCR comprises the amino acid sequences of SEQ ID NO: 6, 7, 8, 9, 10, and/or 11 or an amino acid sequence with at least 60% sequence identity to the amino acid sequences of SEQ ID NO: 6, 7, 8, 9, 10, and/or 11.

47. The engineered TCR of claim 46, wherein the TCR comprises a TCR α CDR3 comprising an amino acid sequence with at least 80% sequence identity to SEQ ID NO:8 and a TCR β CDR3 comprising an amino acid sequence with at least 80% sequence identity to SEQ ID NO:11.

48. The engineered TCR of claim 47, wherein the TCR comprises a TCR α CDR1 and/or CDR2 comprising an amino acid sequence with at least 80% sequence identity to SEQ ID NO:6 and/or 7, respectively and a TCR β CDR1 and/or CDR2 comprising an amino acid sequence with at least 80% sequence identity to SEQ ID NO:9 and/or 10, respectively.

49. The TCR of any one of claims 46-48, wherein the engineered TCR comprises:
(i) an α chain variable region having the amino acid sequence of SEQ ID NO:13 or 2, or a sequence having at least 90% sequence identity to SEQ ID NO: 13 or 2; and/or
(ii) a β chain variable region having the amino acid sequence of SEQ ID NO: 15 or 4, or a sequence having at least 90% sequence identity to SEQ ID NO: 15 or 4.

50. The TCR of any one of claims 46-49, wherein the engineered TCR binds SEQ ID NO:5 when bound to HLA-A2.

51. The TCR of any one of claims 46-50, wherein the TCR comprises an α chain variable region having at least 95% identity to the amino acid sequence of SEQ ID NO: 13 or 2, and/or a β chain variable region having at least 95% identity to the amino acid sequence of SEQ ID NOs: 15 or 4.

52. The TCR of claim 51, wherein the TCR comprises an α chain variable region having at least 99% identity to the amino acid sequence of SEQ ID NO: 13 or 2, and/or a β chain variable region having at least 95% identity to the amino acid sequence of SEQ ID NO: 15 or 4.

53. The TCR of claim 51, wherein the TCR comprises an α chain variable region having at least 95% identity to the amino acid sequence of SEQ ID NO: 13 or 2, and/or a β chain having at least 99% identity to the amino acid sequence of SEQ ID NO: 15 or 4.

54. The TCR of any one of claims 46-50, wherein the TCR comprises an α chain variable region of SEQ ID NO: 13 or 2, and a β chain of SEQ ID NO: 15 or 4.

55. The TCR of any one of claims 46-54, wherein the TCR comprises a modification and/or is chimeric.

56. The TCR of any one of claims 46-55, wherein the soluble TCR is further defined as a single-chain TCR (scTCR), wherein the α chain and the β chain are covalently attached via a flexible linker.

57. The TCR of any one of claims 46-56, wherein the TCR comprises or consists of a bispecific TCR.

58. The TCR of claim 57, wherein the bispecific TCR comprises an scFv that targets or selectively binds CD3.

59. A multivalent TCR complex comprising a plurality of TCRs of any one of claims 46-58.

60. The complex of claim 59, wherein the multivalent TCR comprises 2, 3, 4 or more TCRs associated with one another.

61. The complex of claim 60, wherein the multivalent TCR is present in a lipid bilayer, in a liposome, or attached to a nanoparticle.

62. The complex of claim 60, wherein the TCRs are associated with one another via a linker molecule or a non-naturally occurring disulfide bond.

63. One or more nucleic acid(s) comprising or consisting of a nucleotide sequence encoding the TCR of any one of claims 46-58.

64. The nucleic acid(s) of claim 63, wherein the nucleic acid comprises a cDNA encoding the TCR.

65. An expression vector comprising the nucleic acid of claim 63 or 64.

66. The expression vector of claim 65, wherein the vector comprises the TCR α and TCR β genes.

67. The expression vector of claim 65 or 66, wherein the nucleotide sequence encoding the TCR is under the control of a promoter.

68. The expression vector of any one of claims 65-67, wherein the expression vector is a viral vector.

69. The expression vector of claim 68, wherein the viral vector is a retroviral vector or a lentiviral vector.

70. A host cell engineered to express the TCR of any one of claims 46-58, preferably wherein the host cell comprises the nucleic acid of claim 63 or 64 or the expression vector according to any one of claims 65-69.

71. The host cell of claim 70, wherein the cell is a T cell, NK cell, invariant NK cell, NKT cell, mesenchymal stem cell (MSC), or induced pluripotent stem (iPS) cell.

72. The host cell of claim 70 or 71, wherein the host cell is an immune cell.

73. The host cell of any one of claims 70-72, wherein the host cell is isolated from an umbilical cord.

74. The host cell of any one of claims 71-73, wherein the T cell is a CD8+ T cell, CD4+ T cell, or T6 T cell.

75. The host cell of any one of claims 71-73, wherein the T cell is a regulatory T cell (Treg).

76. The host cell of any one of claims 70-75, wherein the cell is autologous.

77. The host cell of any one of claims 70-75, wherein the cell is allogeneic.

78. A method for engineering the host cell of any one of claims 70-77, comprising contacting the immune cell with the nucleic acid of claim 63 or 64 or the expression vector of any one of claims 65-69.

79. The method of claim 78, wherein the immune cell is a T cell or a peripheral blood lymphocyte.

80. The method of claim 78 or 79, wherein the contacting is further defined as transfecting or transducing.

81. The method of any one of claims 78-80, wherein transfecting comprises electroporating RNA encoding the TCR of any one of claims 46-58 into the immune cell.

82. The method of any one of claim 80 or 81, further comprising generating viral supernatant from the expression vector of claim 68 prior to transducing the immune cell.

83. The method of any one of claims 78-82, wherein the immune cell is a stimulated lymphocyte.

84. The method of claim 83, wherein the stimulated lymphocyte is a human lymphocyte.

85. The method of claim 83, wherein the stimulating comprises contacting the immune cell with or incubating the immune cell in OKT3 and/or IL-2.

86. The method of any one of claims 78-85, further comprising sorting the immune cells to isolate TCR engineered T cells.

87. The method of claim 86, further comprising performing T cell cloning by serial dilution.

88. The method of claim 87, further comprising expansion of the T cell clone by the rapid expansion protocol.

89. A method of treating cancer in a mammalian subject comprising administering an effective amount of the TCR-engineered cells of any one of claims 70-77 to a subject, wherein the cancer expresses Hormad1.

90. The method of claim 89, wherein the TCR-engineered cell is a T cell or peripheral blood lymphocyte.

91. The method of claim 89, wherein the T cell is a CD8+ T cell, NK T cell, iNKT cell, CD4+ T cell, or Treg.

92. The method of any one of claims 89-91, wherein the cancer is a breast cancer, a lung cancer, esophagus carcinoma (esophageal cancer), bone cancer, endometrial cancer, hematopoietic or lymphoid cancer, gastrointestinal cancer, ovarian cancer, skin cancer, neuroblastoma, testicular cancer, thymoma, bladder cancer, uterine carcinoma, melanoma, sarcoma, cervix cancer, head or neck cancer.

93. The method of any one of claims 89-92, wherein the cancer is a solid tumor.

94. The method of any one of claims 89-93, wherein the subject is a human.

95. The method of any one of claims 89-94, wherein the TCR engineered cells are autologous or allogeneic to the subject.

96. The method of any one of claims 89-95, further comprising lymphodepletion of the subject prior to administration of the Hormad1-specific T cells.

97. The method of claim 96, wherein the lymphodepletion comprises administration of cyclophosphamide and/or fludarabine.

98. The method of any one of claims 89-97, further comprising administering a second anticancer therapy to the subject.

99. The method of claim 97, wherein the second therapy is a chemotherapy, immunotherapy, surgery, radiotherapy, or biological therapy.

100. The method of any one of claims 89-97, wherein the TCR-engineered cells, and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

101. The method of any one of claims 89-100, wherein the subject is determined to have or diagnosed as having cancer cells that overexpress Hormad1.

102. An engineered TCR comprising a TCR α chain variable region having a CDR1, CDR2, and CDR3 comprising the amino acid sequence of SEQ ID NO:6, 7, and 8, respectively and a TCR β chain variable region having a CDR1, CDR2, and CDR3 comprising the amino acid sequence of SEQ ID NO:9, 10, and 11, respectively.

103. One or more nucleic acids comprising a cDNA that encodes the TCR α chain variable region and TCR β chain variable region of claim 102.

104. A RNA molecule that encodes both the TCR α chain variable region and TCR β chain variable region of claim 102.

105. A T cell comprising the nucleic acid(s) of claim 103 or the RNA molecule of claim 104.


SEQUENCE LISTING (15 sequences)

SEQ ID NO: 1 - DNA, 804 bp, Artificial sequence
  Synthetic oligonucleotide
atgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtgcgcggggaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 2 - protein, 267 aa, Artificial sequence
  Synthetic polypeptide
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGARGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - DNA, 963 bp, Artificial sequence
  Synthetic oligonucleotide
atgcttagtcctgacctgcctgactctgcctggaacaccaggctcctctgccatgtcatgctttgtctcctgggagcaggttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagccctacgggacagggttcgtacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 4 - protein, 320 aa, Artificial sequence
  Synthetic polypeptide
MLSPDLPDSAWNTRLLCHVMLCLLGAGSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPTGQGSYEQYFGPGTRLTVTEDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 5 - protein, 9 aa, Artificial sequence
  Synthetic peptide
YLDDLCVKI

SEQ ID NO: 6 - protein, 7 aa, Artificial sequence
  Synthetic peptide
NIATNDY

SEQ ID NO: 7 - protein, 5 aa, Artificial sequence
  Synthetic peptide
GYKTK

SEQ ID NO: 8 - protein, 11 aa, Artificial sequence
  Synthetic peptide
LVGARGTALIF

SEQ ID NO: 9 - protein, 5 aa, Artificial sequence
  Synthetic peptide
PRHDT

SEQ ID NO: 10 - protein, 6 aa, Artificial sequence
  Synthetic peptide
FYEKMQ

SEQ ID NO: 11 - protein, 13 aa, Artificial sequence
  Synthetic peptide
ASSPTGQGSYEQY

SEQ ID NO: 12 - DNA, 330 bp, Artificial sequence
  Synthetic oligonucleotide
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtgcgcggggaactgctctgatctttgggaagggaaccaccttatcagtgagttccaat

SEQ ID NO: 13 - protein, 110 aa, Artificial sequence
  Synthetic polypeptide
LAKTTQPISMDSYEGQEVNITCSHNNIATNDYITWYQQFPSQGPRFIIQGYKTKVTNEVASLFIPADRKSSTLSLPRVSLSDTAVYYCLVGARGTALIFGKGTTLSVSSN

SEQ ID NO: 14 - DNA, 342 bp, Artificial sequence
  Synthetic oligonucleotide
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagccctacgggacagggttcgtacgagcagtacttcgggccgggcaccaggctcacggtcaca

SEQ ID NO: 15 - protein, 114 aa, Artificial sequence
  Synthetic polypeptide
AAGVIQSPRHLIKEKRETATLKCYPIPRHDTVYWYQQGPGQDPQFLISFYEKMQSDKGSIPDRFSAQQFSDYHSELNMSSLELGDSALYFCASSPTGQGSYEQYFGPGTRLTVT